(19) United States
(12) Patent Application Publication
(10) Pub. No.: US 2023/0054379 A1
(43) Pub. Date: Feb. 23, 2023

(54) COMPOSITION AND METHOD OF TREATMENT TO SIMULTANEOUSLY INCREASE THE ANALGESIC EFFICACY OF OPIOIDS AND REDUCE THEIR ADDICTIVE POWER

(71) Applicant: Centro de Investigacion Y Desarrollo de Medicamentos (CIDEM), La Habana (CU)

(72) Inventors: Bárbara Beatriz GARRIDO SUÁREZ, La Habana (CU); Pedro Gilberto BÁRZAGA FERNÁNDEZ, La Habana (CU); Alejandro Saúl PADRÓN YAQUIS, La Habana (CU); Yamila VERDECIA REYES, LaHabana (CU); Estael OCHOA RODRIGUEZ, La Habana (CU)

(21) Appl. No.: 17/788,815

(22) PCT Filed: Dec. 18, 2020
(86) PCT No.: PCT/US20/50010

§ 371 Date: Jun. 24, 2022

Related U.S. Application Data
(60) Provisional application No. 63/012,801, filed on Apr. 20, 2020.

(51) Int. Cl. A61K 31/5513; A61K 31/485; A61K 31/135; A61K 31/439; A61K 31/55; A61K 31/4468; A61K 31/454; A61K 31/4535; A61K 31/4465; A61K 31/137; A61K 31/222; A61P 25/04

(52) U.S. Cl. CPC A61K 31/5513; A61K 31/485; A61K 31/135; A61K 31/439; A61K 31/55; A61K 31/4468; A61K 31/454; A61K 31/4535; A61K 31/4465; A61K 31/137; A61K 31/222; A61P 25/04

(57) ABSTRACT

The present invention describes a pharmaceutical composition that includes a novel molecule with neuroprotective activity, to inhibit the neuroadaptations induced by opioids (tolerance and hyperalgesia) that lead to the successive escalation of its doses in the treatment of pain. In this way, it increases its analgesic efficacy in normal conditions and in neural damage, since this favors the appearance of tolerance/hyperalgesia and resistance to opioid treatment. Likewise, reduce the spontaneous signs of withdrawal associated with its withdrawal and consequently physical dependence and possible addiction. In addition, it describes a method for the treatment of pain with a neuropathic component that is supported by drug interaction and safety studies that show its synergy for the mechanical antihypernociceptive effect.

### BACKGROUND OF THE INVENTION

In the absence of a major pain killer discovery, which are indispensable for the treatment of moderate to severe cancer pain, opioids remain the most common drugs prescribed worldwide in the context of the World Health Organization (WHO) analgesic leader, but also for acute and chronic pain management in general (Bennett, N. I. 2012. In: Tracey, I. editor. IASP Refresher Courses on Pain Management. Seattle: IASP Press. p. 301-304; Klepstad, P. et al. 2005. Palliat. Med. 19:477-84; Global market analyses. 2011. In: The Pain Market Outlook to 2016; Business Insights Ltd.: London, UK. p. 50-70). These drugs, which have been classically used for the chronic malignant pain in patients with a short life expectancy, has been extensively introduced in the treatment of chronic non-malignant pain. Despite its recognized adverse effects, several studies show the resurgence around 149% of opioids use during the past two decades. Some of theirs of acute installation such as respiratory depression, and others related to long-term opioid administration such as analgesic tolerance which is associated with successive dose escalation, physical dependence and opioid addition. These practices have facilitated the emergency of a range of complications that increases mortality by over-doses and morbidities such as hypogonadism, osteoporosis, immunosuppression, cognitive impairment and opioid induced hyperalgesia (OIH). Particularity, the use of high doses, which benefits have been hard to demonstrate, have produced disastrous consequences by its addictive potential and a real clinical, social and cultural iatrogenesis. Many of changes introduced in the clinical practice by the palliative medicine which promote the continued exposure to high doses such as the introduction of titrate-to-effect principle with open-ended dose escalation, the idea that opioids should be given at regular intervals, the concept of breakthrough pain and the reformulation of opioids into long-acting preparations, have contributed of these phenomena. On the other hand, currently the major survival of oncologic patients due to immunotherapy, allows to focus the cancer as a chronic non transmissible disease, that it curse with pain and required long-term opioid treatment (Lovejo, T. I. et al. 2017 Pain. 158:526-534; Sullivan, M. D. and Howe, C. Q. 2013. Pain 154: S94-S100; Rivat, C. and Ballantyne, J. 2016. Pain Reports 1: e570; Petersen, K. L. et al. 2008. Pain. 137:395-404; Boudreau, D. et al. 2009. Pharmacoepidemiol. Drug Saf.18:1166-75; Raghavan, S. et al., 2011. Trends in Anaesthesia and Critical Care. 1:18-21).

OIH is a fact not exclusively provoked by morphine, if not by other drugs from this group with preponderant activity on μ receptor such as fentanyl, remifentanil, sufentanil which are recognized by its analgesic potency, but low pharmacologic profile. In addition, codeine, one of the weak opioid most utilized, is metabolized to morphine in 10% and also shows this complication. Opposed to buprenorphine, a partial μ agonist and κ, δ, antagonist, which is a weak analgesic but shows anti-hyperalgesic properties that it mainly attributed to its κ antagonism. Then, at the problems as high incidence and resistance of cancer pain it add the neurobiological adaptations to analgesia (tolerance and hyperalgesia) induced by the most potent analgesics used in its relief. These neuroadaptations interfere with the ability of opioids to induce a long-term analgesia and induce a latent sensitization of pain with adaptive changes that lead to its chronicity, with the subsequent clinical repercussion. While these paradoxical phenomena also have been reported after a short-term potent opioid administration on anaesthetic-surgical act with pain increases as well as of the opioids requirement for its post-operative treatment. Thence, a strategy that permit minimize these changes, in particular during long-term therapy as need the contemporary oncologic patients, is necessary (Rivosecchi, R. M. et al. 2014. Expert Opin. Drug Saf.13:587-603; Angst, M. S. et al. 2003. Pain. 106:49-57; Gardell, L. R. et al. 2002. J Neurosci. 22:6747-6755; Laughlin, T. M. et al. 1997. Pain. 72:253-260).

Tolerance involves a “within process” adaptation, in which drug administration elicits an opposing reaction within the same system in which the drug elicits its primary action. Such an adaptive response progressively neutralizes the drug's effect, exemplified by mechanisms of opioid tolerance that involve desensitization. OIH is referred to as “between process” adaptation, which is based on the notion that drug administration recruit different neuronal circuits that oppose the primary drug effect, in special of specific pronociceptive signalling as the glutamatergic by means of the engage of N-methyl-D-aspartate (NMDA) receptors. Indeed, blocking NMDA receptors can prevent the development of tolerance by decreasing the activation of pronociceptive systems that are triggered by opioids. This engagement of the second process may result in an opioid-induced increase of pain sensitivity (allodynia and hyperalgesia) induced by opioid. Tolerance is characterized by a progressive lack of response to morphine that can be overcome by increasing the dose, whereas hyperalgesia is a sensitization process by which opioids, paradoxically, cause pain hypersensitivity during its administration and/or after its discontinuation Both mechanisms lead to the decreased efficacy of opioid analgesic effects, discontent of patients which would need increased opioid doses (Xie, J. Y. et al. 2005. J Neurosci. 25:409-16; Wang, Z et al. 2010. Pain 151:194-205; King, T. et al. 2005. Pain 116:276-288; Duttaroy, A. et al. 1999. Life Sci. 65:113-23; Aira, Z et al. 2012. Pain 153:1418-1425; Gardell, L. R. et al. 2002. J Neurosci. 22:6747-6755; Laughlin, T. M. et al. 1997. Pain. 72:253-260; Roeckel, L. A. et al. 2016. Neuroscience. 338:160-182). In general, today exists a growing cumulus of evidence which suggest that the develop of OIH is mediated by neural mechanisms that involve cellular and molecular changes in neural networks that also are implicate in neuropathic pain (NP) establishment. The resistance to opioids in patients with neural injury is a solid clinic element that claim this motion, as well as the evidences that demonstrates the involvement of glial cells networks from the microglia and astrocytes activation, pro-inflammatory cytokines expression and aberrant function of glutamate transporters (GTs) in both paradigms. Consequently, to the classic neural mechanisms of adaptation and sensitization, the tetrapartite synapse mechanisms that actively involve the neuron-glial cells interactions in the central sensitization process and in OIH, are added at present (Mayer D. J. et al. 1999. Proc Natl Acad Sci USA. 96:7731-7736; Manning, D. C. 2006: In: Campbell, J. N, Basbaum, A. I., Dray, A., Dubner, R, Dworkin, R. H, Sang, Ch. N. Emerging Strategies for the Treatment of Neuropathic Pain, IASP Press, Seattle, p. 161-192; Tawfik V L, and De Leo J. 2007. Modulating glial activation in opioid tolerance and neuropathic pain: A role for glutamate transporters. In De Leo J A, Sorkin L S, Watkins L R. Immune and glial regulation of pain. IASP Press, Seattle, p 341-359; De Leo, J. A. et al. 2006. Pain 122:17-21).

Opioids exert their analgesic effects through binding the opioid family of classic seven-transmembrane-spanning G-protein-coupled receptors (μ, κ, δ) specifically of the inhibitory complex Gi/o. These receptors are further linked to an inwardly rectifying potassium channel (Kir) of the family of potassium channel sensitive to ATP and therefore, ligand binding leads to hyperpolarization of the pre-synaptic and post-synaptic membrane neurons which are responsible in part of its analgesic effects, in addition to the inhibition of calcium channels, sodium channels and the adenylate cyclase enzyme. However, opposite, opioids have ability to activate the pro-nociceptive signalling pathway phospholipase C β (PLCβ)/protein kinase Cγ (PKCγ) inositol-lipid through βγ subunit of Gi protein which promote the release of endogenous Ca²⁺ and the translocation/activation of PKC. Then this facilitate the removal of Mg²⁺ from NMDA receptors which respond to glutamate released from the pre-synaptic endings rapidly boosting synaptic efficacy and allows entry of Ca²⁺ into the neuron, which then activates numerous intracellular pathways, particularly PKC that lead to increases the phosphorylation of NMDA receptors. In addition to G protein, opioid agonist can activate parallel or distinct signalling pathways. G protein-coupled receptor kinases (GRK) phosphorylate to receptor promoting the translocation of β-Arrestin to receptor and inhibit its activation interrupting its coupled with G protein. β-Arrestin binding to the μ opioid receptor induces internalization, desensitization and this specific signalling is considered involved in opioid-induced respiratory depression, constipation, analgesic tolerance, OIH, physical dependence and addiction. On the other hand, opioids also can also interact via μ receptors present in microglia and astrocytes, which are activate in presence of morphine, then a neuroimmune mediation of tolerance/OIH take place. Regarding to this, an increases of glial fibrillary acidic protein (GFAP) in the spinal cord, posterior cingulate cortex and hippocampus following chronic intraperitoneal treatment of rats with morphine, as well as, the partial restored of morphine analgesia after the block of astrocyte hyperactivity using the glial metabolic inhibitor fluorocitrate, has been reported. GFAP and OX-42 immunoreactivity as well as enhanced expression of IL-1β, IL-6 and TNFαwere observed only after chronic, but not acute morphine administration. Although there are reports on the increase of the protein expression of IL-1β from the satellite cells in the dorsal root ganglia (DRG) through the activation of matrix metalloproteinase-9 (MMP-9) after the acute and chronic administration of morphine. In general, at cellular level, neurons and glial cells shows synergic effects that contribute to 01H and in this context, several molecular actors have been identified such as Toll-like receptor 4 (TLR4), anti-opioid system, excitatory molecules, receptors, channels, chemokines, pro-inflammatory cytokines and lipids. One of the most prominently reported cascades activated by opioid exposure is the MAP kinase pathway which are a collection of serine/threonine-specific protein kinases [p38, c-Jun N-terminal kinase (JNK), and extracellular signal regulated kinase (ERK)] as well as, the mammalian target of rapamycin (mTOR). Particularly, morphine produces phosphorylation of p38 within microglia (Mayer D. J. et al. 1999. Proc Natl Acad Sci USA. 96 :7731-7736; Williams, J. T. et al. 2013. Pharmacol Rev. 65:223-54; Galeotti, N. et al. 2006. Pain 123:294-305; Raghavendra, V. et al. 2002. J. Neurosci. 22:9980-9989; Sanna, M. D. et al. 2015. Pain 156:1265-127; Johnston, I. N. et al. 2004. J. Neurosci. 24:7353-65; Chen, X. et al. 2007. Brain Res 2007; 1153:52-7; Vardanyan, A. et al. 2009. Pain 10:243-52; Roeckel, L. A. et al. 2016. Neuroscience. 338:160-182; Muscoli, C. et al. 2010. J. Neurosci. 30:15400-8; Cui, Y. et al. 2008. Brain Behav lmmun. 22:114-23; Wang, Z et al. 2010. PAIN 151:194-205; Xu, J. T. et al. 2014. J Clin Invest. 124:592-603; Gwak, Y. S. and Hulseboch, C. E. 2011. Neuropharmacology 60: 799-808; Hasanein, P. et al. 2008. Brain Res. 1241:36-41; Möhler, H. 2011. Neuropharmacology 60: 1042-1044; De Leo, J. A. et al. 2006. Pain 122:17-21; Trang, T. et al. 2015. J Neurosci. 35:13879-13888; Tsuda, M. et al. 2003. Nature 424:778-83; Horvath, R. J. et al. 2010. PAIN 150:401-413; Berta, T. et al. 2012. Mol. Pain 8:18.; Nakamoto, K et al. 2012. Eur J Pharmacol. 683:86-92). Besides, spinal mitochondrial-derived peroxynitrite (ONOO-, PN) enhances neuroimmune activation during OIH. PN can cause mitochondrial dysfunction by suppress mitochondrial respiration, promote apoptosis, and increase reactive oxygen species generation by inactivating electron transport complexes I, II, III, and V; nitrating and inactivating aconitase; nitrating and activating cytochrome c; and inducing opening of the membrane permeability transition pore. Other element that contribute to OIH and tolerance anti-nociceptive is the nitrating and inactivating of the mitochondrial manganese superoxide dismutase (MnSOD) enzyme. PN is also required for NF-κB, ERK, and p38 activation as well as enhanced levels of pro-inflammatory cytokines in these conditions. This reciprocal regulation between cytokines and PN has also been defined at spinal level during the development of pain of different aetiologies, including NP induced by chemotherapeutic agents such as paclitaxel. The disruption of GTs caused by PN increases glutamatergic signalling, particularly by nitration and inactivation of the glutamate transporter-1 (GLT-1), glutamate-aspartate transporter (GLAST) and excitatory amino acid channel 1 (EAAC1), as well as the glutamine synthetase (GS) that converts glutamate, ammonia, and ATP to glutamine, which is then taken back up by the neurons via the glutamine transporter. In addition, EAAC1 transports cysteine to the neurons, which is decisive for the synthesis of glutathione (GSH), one of the most important cellular non-enzymatic systems. Hence, nitroxidative stress and mitochondrial dysfunction are proposed as sources of therapeutic targets, in particular PN, to improve the efficacy of morphine. At the preclinical level it has also been suggested that group I metabotropic glutamate receptor (mGluR5) antagonists and group II (mGluR2/3) and III (mGluR7) agonists decrease morphine tolerance under conditions of neural injury. In addition, the attenuation of OIH by dexamethasone has been reported in relation to an increase in GTs and a reduction in excitatory amino acids. Consequently, the stabilization of astrocytes for their competence in maintaining excitatory amino acid homeostasis is considered another strategy that is currently exploited (Little, J. W. et al. 2013. PAIN 154:978-986; Doyle, T. et al. 2010. Neurosci. Lett. 483:85-9; Muscoli, C. et al. 2004. Pain 111:96-103; Muscoli, C. et al. 2007. J Clin Invest. 117:3530-9; Ndengele, M. M. 2009. J PharmacoL Exp. Ther. 329:64-75; Chen, Z et al. 2010. Pain 149:100-106; Salvemini, D. et al. 2011. Free Radical Bio. Med. 51:951-966; Janes, K. 2013. Pain 154:2432-40; Osikowicz, M. et al. 2008. Pain 139:117-126; Tai, Y. H. et al. 2007. Pain 129:343-354). Furthermore, interestingly the exacerbation of these neuroadaptations have been demonstrated after the administration of chronic morphine in rats with lesions of the L5 spinal nerve or of the sciatic nerve, a finding that suggests the facilitation of OIH in conditions of neural injury (Tawfik V L, and De Leo J. 2007. Modulating glial activation in opioid tolerance and neuropathic pain: A role for glutamate transporters. In DeLeo J A, Sorkin L S, Watkins L R. Immune and glial regulation of pain. IASP Press, Seattle, p 341-359; Christensen, D. and Kayser, V. 2000. Pain 88:23-238; Sung, B. et al. 2003. J Neurosci. 23:2899-2910; De Leo, J. A. et al. 2006. Pain 122:17-21).

Some pharmacological strategies for the development of more effective and safe opioids are directed towards the search for agonists of the G-protein-biased μ-opioid receptor such as TRV130 that activates the pathway of the protein G responsible for analgesic activity, but blocks the pathway of β-arrestin. Another option has been the development of multifunctional drugs with mixed opioid and non-opioid activity, particularly peptide mimetic ligands that combine potent μG agonistic activity with antagonist activity of the FF1 and 2 (NPFF1/2) neuropeptide receptors involved in OIH and other adverse effects. On the other hand, clinical strategies to face OIH have focused on the simultaneous administration of glutamatergic receptor antagonists, the rotation of different opioid medications (due by their inter-individual differences in the ability to produce activation, desensitization and endocytosis) or the combination of opioid agonists with low-dose antagonists. As well as, combined opioid therapies with adjuvants or co-analgesics such as clonidine, parecoxib, propofol, pregabalin, midazolam or other opioids with multimodal actions such as methadone to optimize treatment. However, NMDA receptor antagonists such as ketamine have been associated with serious adverse effects for their long-term use, particularly on the cognitive sphere and impaired memory. The contribution of β₂ adrenergic receptors to the remifentanil-induced postinfusion hyperalgesia and its reduction by propranolol have also been reported. In particular, the beneficial effect of the co-administration of ultra-low doses of naloxone in tolerant rats has been related to the attenuation of NMDA neurotransmission and the suppression of spinal cord neuroinflammation (Chu, L F et al. 2012. Pain 153:974-981; Mao, J. 2002. Pain 100: 213-217; Lin, S L. 2010. Pharmacol. Biochem Behav 96:236-245; Drieu la Rochelle, A et al. 2018. Pain 159:1705-1718; Loftus R W, et al. 2010. Anesthesiology 113:639-46; Bannister, K. 2015. Curr Opin Support Palliat Care 9:116-121; Reddy, A et al. 2014. Expert Opin. Drug Saf.14(1)). JM-20 (3-ethoxycarbonyl-2-methyl-4-(2-nitrophenyl)-4,11-dihydro-1H-pyrido [2,3-b] [1,5] benzodiazepine) is a hybrid molecule that has been studied in in vitro, ex-vivo and in vivo relevant models for cerebral ischemia. This differs from the known 1,5 benzodiazepines (BDZ) by the fusion of a 1,4 dihydropyridine. This molecule exhibits potent neuroprotective effects related to its ability to decrease glutamatergic signalling, neuroinflammation, mitochondrial dysfunction and apoptosis in these conditions. The results suggest its possible interaction with the heteropentameric chloride-permeable ion channels GABA_{A} receptor complex which is considered the central benzodiazepine receptor (CBR) and with voltage-gated calcium channel (VGCC), particularly those of L type. The observation of neurological behaviors related to GABAergic signaling in different animal models have suggested that the dihydropyridine portion does not interfere with this effect. Consequently, it has been protected structurally and for the treatment of diseases of the nervous and vascular system in WO/2011/041989. Subsequently, this compound, its derivatives and the pharmaceutical compositions that contain it were protected for the specific treatment of neurodegenerative disorders, with cognitive impairment, diseases associated with oxidative stress, diseases that occur with mitochondrial dysfunction, Parkinson's disease and neuropathic pain, as well as pathologic processes associated with aging in WO/2017/190713. In particular, JM-20 in combination with the phenolic compound KM-34, which has a greater antioxidant power, was protected in WO/2017/190714 for the treatment of ischemic cerebrovascular disease. JM-20 has been reported to prevent glutamate and hydrogen peroxide-induced cell death or KCN-induced chemical hypoxia. JM-20 is able to preserve its neuroprotective effect in primary cerebellar cell cultures exposed to glutamate plus pentylenetetrazole (PTZ), an agent that interacts with the picrotoxin site of postsynaptic GABAA receptors to exert a non-competitive antagonism with GABA and exacerbate NMDA receptor-mediated toxicity. Which suggests not only its power but also its multimodal mode of action. JM-20 treatment also protected brain mitochondria from ischemic insult through prevention of mitochondrial transition permeability, swelling, membrane potential dissipation and organelle release of the pro-apoptotic protein cytochrome c, as well as the succinate-mediated generation of H₂O₂, the uncoupling of respiration and the influx of Ca²⁺ into the mitochondria. Furthermore, this molecule reduces the concentrations of glutamate and aspartate in the cerebrospinal fluid and the deleterious effects of the middle cerebral artery occlusion (MCAo). JM-20 modulates glutamatergic neurotransmission through the reduction of H⁺-ATPase activity and vesicular glutamate reuptake, consequently, it inhibits the neuronal release of this neurotransmitter, associated with the increase of its recapture by the astrocyte, thus promoting glutamate homeostasis in the brain. On the other hand, this compound exerts its actions against ischemic insult through mechanisms that involve the modulation of reactive astrogliosis, as well as neuroinflammation and the anti-apoptotic cell signaling pathway. JM-20 reduces cell death and prevents oxygen and glucose deprivation (OGD)-induced phosphorylated Akt decrease and GSK-3p dephosphorylation in hippocampal cells. The effect is associated with the reduction of caspase-3 activation and the suppression of microglial activation with the consequent decrease in pro-inflammatory cytokines such as interleukin (IL)-1β, IL-6, tumor necrosis factor alpha (TNF-α) and increased levels of the anti-inflammatory cytokine IL-10 (Núñez-Figueredo, Yet al. 2013. Neurol Res 35: 804-12; Núñez-Figueredo, Y et al. 2014. Neuropharmacology 85: 517-27; Núñez-Figueredo, Y et al. 2014. Brain Res Bull 109: 68-76; Núñez-Figueredo, Y et al. 2014. Eur J Pharmacol 726: 57-65; Núñez-Figueredo, Yet al. 2015. Neurochem Int 81: 41-7; Ramirez-Sánchez, J et al. 2015; Neurochem Int 90: 215-223; Ramirez-Sánchez, J et al. 2018. Mol Neurobiol https://doi.org/10.1007/s12035-018- 1087-8)

It has now been surprisingly found that JM-20, a compound that can modulate glutamatergic signaling (without blocking NMDA receptors), neuroinflammation and mitochondrial dysfunction in models of cerebral ischemia, shows the ability to attenuate the development of tolerance and hyperalgesia. induced by opioids, such as morphine. Furthermore, JM-20, which has shown a selective mechanical antihypernociceptive effect in NP models, when associated with morphine improves its efficacy in conditions of neural injury. In particular, the introduction of JM-20 associated with opioids such as morphine is proposed for the multimodal treatment of pain, where the opioid treatment in the short or long term also induces neuroadaptations that interfere with its analgesia efficacy and promote latent sensitization of the pain.

### BRIEF DESCRIPTION OF THE INVENTION

The present invention relates to a combination therapy comprising a first composition a) comprising the JM20 compound and a second composition b) comprising at least one opioid. A particular aspect of the present invention is a combined therapy comprising compositions a) and b) at fixed doses. Particular forms of embodiment would be the presentation of this combination in a single pharmaceutical form, packaged in a single package or in separate packages. Another aspect of the present invention refers to the use of the pharmaceutical combination to prevent analgesic tolerance and hyperalgesia induced by short and long-term exposure to opioids, as well as physical dependence, withdrawal symptoms and some factors (agent and phenomena pharmacological) that influence abuse and addiction due to repeated use. One particular aspect concerns the treatment of morphine-resistant neuropathic pain. Another aspect of the present invention relates to a method of treating a patient who may develop opioid-induced neuroadaptations which comprises administering to the patient a therapeutically effective amount of the combination therapy to enhance the analgesic efficacy of the opioid and reduce dependency, symptoms withdrawal and addictive power. One particular aspect relates to a method of treating a patient with neuropathic pain receiving opioid treatment who may be more susceptible to develop the OIH.

### BRIEF DESCRIPTION OF THE FIGURES

FIG. 1. Effect of co-treatment with JM-20 on the induction of mechanical allodynia (A) and mechanical hyperalgesia (B) by chronic morphine treatment in rats, evaluated by the response percentage of the hind paws to the von Frey 4 g filament and 15 g respectively.

FIG. 2. Effect of co-treatment with JM-20 on the intensity of mechanical (A) and thermal (B) hypernociception induced by chronic morphine treatment in rats expressed as (A) force in grams and withdrawal latency of the paw in the hot plate test, respectively.

FIG. 3. Effect of co-treatment with JM-20 on the establishment of hyperalgesia induced by chronic morphine treatment in rats evaluated through the weighted formalin pain score in this test.

FIG. 4. Effect of co-treatment with JM-20 on the appearance of symptoms of spontaneous withdrawal in tolerant/hyperalgesic rats after chronic morphine administration on day 1 of withdrawal. A. Jumping or escape attempts. B. Paw tremor. C. Mastication. D. Piloerection, E. Shaking in wet dog.

FIG. 5. Time course of the effect of an acute systemic dose of morphine on the intensity of mechanical hypernociception in mononeuropathic animals after 4 days of pre-treatment with saline (open circles) or morphine (solid circles) (A) and treatment with JM- 20 or your vehicle for 5 days in these conditions. The measurement was carried out at 19 hours after the last subcutaneous injection and 6 hours after the oral one.

FIG. 6. Mechanical antihypernociceptive effect (A) of increasing doses of morphine administered subcutaneously and expressed as (Δ) force in grams. These results expressed as a percentage of the maximum possible effect (% MPE) (B) in the ipsilateral paw of CCI rats.

FIG. 7. Mechanical antiallodynic effect (A) of increasing doses of morphine administered subcutaneously and expressed as 50% of the withdrawal threshold for von Frey filaments. These results expressed as a percentage of the maximum possible effect (% MPE) (B) in the ipsilateral paw of CCI rats.

FIG. 8. Thermal antihypernociceptive effect (A) of increasing doses of morphine administered subcutaneously and expressed as paw withdrawal latency in the Hargreaves test. These results expressed as a percentage of the maximum possible effect (% MPE) (B) in the ipsilateral paw of CCI rats.

FIG. 9. Mechanical antihypernociceptive effect (A) of increasing doses of JM-20 administered orally and expressed as (A) force in grams. These results expressed as a percentage of the maximum possible effect (% MPE) (B) in the ipsilateral paw of CCI rats.

FIG. 10. Mechanical antiallodynic effect (A) of increasing doses of JM-20 administered orally and expressed as 50% of the withdrawal threshold before von Frey filaments. These results expressed as a percentage of the maximum possible effect (% MPE) (B) in the ipsilateral paw of CCI rats.

FIG. 11. Thermal antihypernociceptive effect (A) of increasing doses of JM-20 administered orally and expressed as paw withdrawal latency in the Hargreaves test. These results expressed as a percentage of the maximum possible effect (% MPE) (B) in the ipsilateral paw of CCI rats.

FIG. 12. Mechanical antihypernociceptive effect (A) of increasing doses of the drug-therapeutic combination morphine-JM-20 expressed as (Δ) force in grams. These results expressed as a percentage of the maximum possible effect (% MPE) (B) in the ipsilateral paw of CCI rats.

FIG. 13. Mechanical antiallodynic effect (A) of increasing doses of the drug-therapeutic combination morphine-JM-20 expressed as 50% of the withdrawal threshold for von Frey filaments. These results expressed as a percentage of the maximum possible effect (% MPE) (B) in the ipsilateral paw of CCI rats.

FIG. 14. Thermal antihypernociceptive effect (A) of increasing doses of the drug-therapeutic combination morphine-JM-20 expressed as paw withdrawal latency in the Hargreaves test. These results expressed as a percentage of the maximum possible effect (% MPE) (B) in the ipsilateral paw of CCI rats.

FIG. 15. Mechanical antihypernociceptive effect of increasing doses of morphine (A), JM-20 (B) and the drug-therapeutic combination morphine-JM-20 (C) evaluated at the time of the first administration at 7 days and after repeated doses at 14 days post-CCI expressed as (Δ) force in grams in the ipsilateral paw of CCI rats.

FIG. 16. Isobolographic analysis showing the synergistic interaction of the drug-therapeutic combination morphine-JM-20 by subcutaneous and oral routes respectively at a single dose after 7 days of induction of chronic constriction injury of the sciatic nerve in rats.

FIG. 17. Qualitative histopathological analysis of sciatic nerve sections from rats to evaluate the neuroprotective effect of JM-20 (10mg/kg) on the changes related to Wallerian degeneration induced by sciatic nerve chronic constriction injury (CCI) 14 days' post-surgery exacerbated by chronic morphine treatment.

### DETAILED DESCRIPTION OF THE INVENTION

In a study of 3030 patients affected by cancer pain, morphine was reported as the most widely used opioid for the treatment of moderate to severe pain, 40% orally and 10% parenterally, followed by fentanyl patches (14%), oxycodone (4%), methadone (2%) and hydromorphone (1%). These powerful analgesics are associated with the phenomena of tolerance and hyperalgesia that lead to a reduction in their efficacy and an increase in therapeutic doses. In the present invention related to the co-administration of the neuroprotective multimodal compound JM-20 with opioid drugs, in this case morphine, its ability to prevent the onset of tolerance induced during the administration of the opioid and hyperalgesia after its discontinuation or withdrawal was confirmed in healthy and mononeuropathic rats. Likewise, a reduction in spontaneous withdrawal signs was observed after the withdrawal of morphine. This strategy provides advantages compared to others in the state of the prior art, such as the association of opioids with NMDA antagonists. These drugs have been associated with serious adverse effects that invalidate their long-term use, particularly in the cognitive sphere. Long-term potentiation (LTP) at the spinal level is one of the forms of persistent plasticity associated with central sensitization, but in the brain it is a process of synaptic enhancement that participates in the storage of memory in the hippocampus. However, there is a crucial difference between brain LTP and spinal LTP, precisely because glial activation and the consequent production of pro-inflammatory cytokines inhibit LTP in the hippocampus, but promote spinal LTP (Liu, X G and Zhou, L J 2015. Current Pharmaceutical Design 21, 895-905). The JM-20 aimed at stabilizing the function of microglia and astrocytes, reducing neuroinflammation, and improving glutamate homeostasis, could not only attenuate opioid-induced painful latent sensitization, but also favor memorization processes.

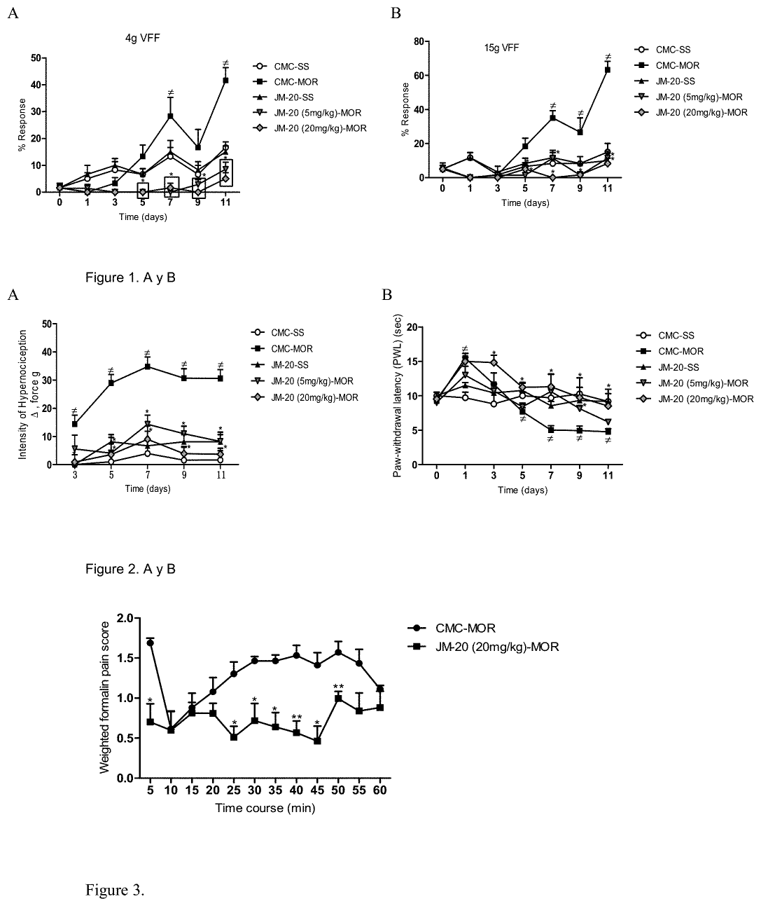

The experiments were designed using established models of induction of tolerance to morphine and hyperalgesia upon withdrawal. Young rats (8-10 weeks) Sprague Dawley (SD) (males, 168-240 g) from the National Center for the Production of Laboratory Animals (CENPALAB, Havana, Cuba) were used, adapted for seven days to the conditions of the laboratory. The animals were kept under an alternating cycle of light and dark every 12 h, the temperature was controlled between 25-27° C. and an EMO 1001 diet established for the laboratory animals. After the adaptation period, the animals were distributed in the different groups according to their body weights. Water and food were kept available ad libitum. All procedures were performed according to the European regulations for the protection of animals (Directive 86/609), the declaration of Helsinki, and/or the Guidelines for the Care and Use of Laboratory Animals adopted and promulgated by the US National Institute of Health. (NIH Publication No. 85-23, revised 1996). All the experimental protocols were approved by the Ethics Committee for Animal Experimentation of CIDEM, Havana, Cuba. To induce tolerance/hyperalgesia to morphine, it was administered to the animals subcutaneously (10 mg/kg, 1 mUkg, s.c.) twice a day every 12 h for 10 days or saline solution (Cao, J L et al. 2005 Pharmacol Biochem and Behay. 80: 493-503), after oral administration of JM-20 or its vehicle, carboxymethylcellulose (0.5% CMC in distilled water, 10mUkg, p.o.) 1h before. Correspondingly, the following groups were constituted: CMC-SS (n=6), CMC-MOR (n=6), JM-20-SS (n=6), JM-20 (5mg/kg)-MOR (n=6) and JM-20 (20mg/kg)-MOR (n=6). At 45 minutes after morphine administration, estimates were made to determine the presence of sensory alterations (mechanical allodynia and hypernociception). The evaluation of the mechanical afferent systems was performed by using von Frey filaments (VFF) (Stoelting nylon monofilaments, Woodale, IL) and an electronic von Frey model INSIGHT®, Brazil, respectively, while the sensitivity of the thermal afferent systems was evaluated using the hot plate test. The evaluations were carried out under baseline conditions and from days 1, 3, 5, 7 and 9 during the medication to show the induction of tolerance, with the exception of the measurement of mechanical hypernociception that began from the 3rd day. Established hyperalgesia (hypernociception in animals) was evaluated in a similar way, but at 11 days in the absence of drug administration. At this same time point, another group of animals that expressed 01H to the thermal stimulus, were exposed to the formalin test. They received a test dose of morphine (5mg/kg) intraperitoneally 20 minutes prior to the administration of intraplantar 5% formalin. In order to assess their responsiveness to an acute dose of morphine, nocidefensive behaviors were observed for 1 h, according to the re-modified method of Dubuison and Dennis (Watson, G S et al. 1997. Pain 70: 53-8). The undamaged animals exposed to the chronic treatment of morphine showed a higher percentage of response to the von Frey filaments 4 g, 8 g and 15 g from day 7 with respect to their controls treated with saline. FIGS. 1A and B show the results that indicate the presence of mechanical allodynia (VFF 4 g) and mechanical hypernociception (VFF 15 g). The differences in the intensity of hypernociception evaluated by means of the withdrawal threshold (Δ) in grams with respect to the baseline values were observed from day 3 of exposure to morphine, as well as the withdrawal latency of their paws (PWL) showed trend towards reduction from this moment on and a significant reduction from day 5 (FIGS. 2.A and B). The changes in all modalities were very evident by day 11 after withdrawal, as previously described. Particularly when exposed to a chemical irritant in the 5% formalin test, the tolerant/hyperalgesic animals showed a lower response to the acute dose of morphine, which was evidenced by the higher values of their nociceptive scores with respect to the animals treated with SS s/c for 10 days. Co-treatment with JM-20 in this paradigm was effective in preventing morphine-induced tolerance and hyperalgesia phenomena. Animals treated with JM-20 significantly reduced the percentages response to the 4 g VFF and 15g VFF, as well as the value of the withdrawal threshold (Δ). This treatment prevented thermal hyperalgesia (decrease in PWLs before thermal stimulation) due to morphine treatment at 11 days and significantly improved nociceptive scores in the formalin test in both phases. Previously, we had observed a selectivity of JM-20 to reduce mechanical hypernociception in mononeuropathic animals, in which this molecule was ineffective in the Hargreaves test (Hargreaves, K. et al. 1988. Pain 32: 77-88). It has been reported that the mechanical stimulation modality activates the inhibitory spinal neurons more vigorously than the thermal and that the mediation of the GABAA receptor in the modulation of sensitivity to mechanical stimuli is clearly manifest, while the modulation of thermal stimuli is less pronounced (Furue, H. et al. 1999 J. Physiol. 521 (Pt. 2): 529-535; Anseloni, V C and Gold, M S 2008. J. Pain 9: 732-738). Anatomical evidence suggests that low-threshold mechano-receptors receive more pre-synaptic GABAergic impulses, therefore, the selectivity in the inhibition of this sensory modality suggests that the benzodiazepine fraction of this hybrid compound could be involved in this effect through spinal GABAA receptors implicated in the modulation of these reflex behaviors evaluated in rodents (Todd, A J 2002. Exp. Physiol 87: 245-249; Price, T J et al. 2009. Brain Res Rev 60: 149-170). The GABA_{A} receptors containing the γ² subunit, which are sensitive to BDZ, are predominantly located in synapses. This evidence supports some of the theories that explain the analgesic actions of these compounds in conditions of inflammation or neural injury in which a depolarizing change in the anion equilibrium potential (E anion) with an impact on GABAergic inhibition has been demonstrated. GABA_{A} receptors of extra-synaptic location that do not contain this subunit, could be protagonists in the depolarizing change in the E anion that promotes the pronociceptive action of the activation of GABAA receptors and disinhibition (Johnston, G A 2005, Curr. Pharm. Des. 11: 1867-1885; Asiedu, M. et al., 2010. Pain 148: 302: 308; Coull, J A et al., 2003. Nature 424, 938-942; Coull, J A et al., 2005. Nature 438: 1017-1021). The results on the analgesic profile of JM-20 suggest its preponderant activity in conditions of alteration of nociceptive processing, but not in non-injured animals where the nociceptive response it is normal. Some of the mechanisms underlying hyperalgesia following inflammation or neural damage are also actors in OIH. In this experiment, the animals of the JM-20-SS group exposed to mechanical and thermal stimuli did not show differences with respect to the animals of the control group (CMC-SS). The ability of midazolam to prevent the establishment of tolerance/OIH in healthy rats had previously been described (Cao, J. L. et al. 2005. Pharmacol Biochem and Behay. 80: 493-503). Which was related to the inhibition of Fos protein expression in superficial and deep lamina neurons of the contralateral spinal dorsal horn (SDH), as well as with the reduction of the activity and expression of the neuronal nitric oxide synthase enzyme (NOSn) and inducible (NOSi) spinal. Furthermore, in in vitro studies it has been reported that this BZD can suppress the release of NO and TNF-α induced by LPS in activated microglia (Wilms, H. et al. 2003. Neurobiol Dis. 14:417-24.). The BDZ portion of JM-20 is active and could prevent tolerance/hyperalgesia in these animals by similar mechanisms, interacting through central or peripheral BZD receptors, the first ones coupled to GABA_{A} are expressed in astrocytes and PRBs increase their expression in activated microglia and astrocytes in conditions of neural injury, ischemia or inflammation, and constitute part of the mitochondrial permeability transition pores (mPTP) complex (Bordet, T. and Pruss, R M. 2009. Neurotherapeutics 6: 648-662). PN derived from spinal mitochondria increases neuroimmune activation during OIH and can cause mitochondrial dysfunction, so the mitoprotective ability and to modulate neuroinflammation of this hybrid molecule could also be involved in the preventive capacity of OIH in rats (Little, J W et al. 2013. PAIN 154: 978-986; Núñez-Figueredo, Yet al. 2014. Neuropharmacology 85: 517-27; Núñez-Figueredo, Yet al. 2014. Brain Res Bull 109: 68-76; Núñez-Figueredo, Y et al. 2014. Eur J Pharmacol 726: 57-65; Ramirez-Sánchez, J et al. 2015; Neurochem Int 90: 215-223; Ramirez-Sánchez, J et al. 2018. Mol Neurobiol https://doi.org/10.1007/s12035-018-1087-8).

The association of opioid drugs and benzodiazepines is not a safe combination due to the risk of additivity of the effects of sedation and respiratory depression of both drugs, so it is not recommended in clinical conditions (Sullivan, M. 2018. Pain 159: 407— 408). Furthermore, it has been reported that the chronic administration of BDZ may also produce adaptive responses in the central nervous system (CNS) such as tolerance to its anxiolytic effect and physical dependence (Vinkers, C H and Oliver, B. 2012. Adv Pharmacol Sci https://10.1155/2012/ 416864). Despite its genuine analgesic effects, addictive tendency, sedation, and cognitive decline are some of the adverse effects that contraindicate the long-term use of BDZ for the treatment of chronic pain. During the pharmacological safety tests carried out in the course of these experiments, exclusively after the first dose of subcutaneous morphine, the latency time for the fall of the animals in the rotary rod test was reduced as a consequence of its sedative effect. However, it has been surprisingly found that the animals co-treated with JM-20, although different from those of the control group (CMC-SS), presented significantly higher latencies than the CMC-MOR group, so that their association at doses of 20 mg/kg with MOR did not constitute a risk for sedation and respiratory depression, but rather a protective element (Tables 1, 2 and 3). This effect could be attributed to the activity of the dihydropyridine portion of this molecule, with possible supra-additive actions for the prevention of neuroadaptations as has been reported for nimodipine, but also to reduce some of the acute adverse effects of morphine (Zharkovsky, A. et al. 1993. Naunyn Schmiedebergs Arch Pharmacol 347: 483-486; Bernstein, M A and Welch, SP 1995. Brain Res 696: 83-88; Drieu la Rochelle, A et al. 2018. Pain 159: 1705 -1718).

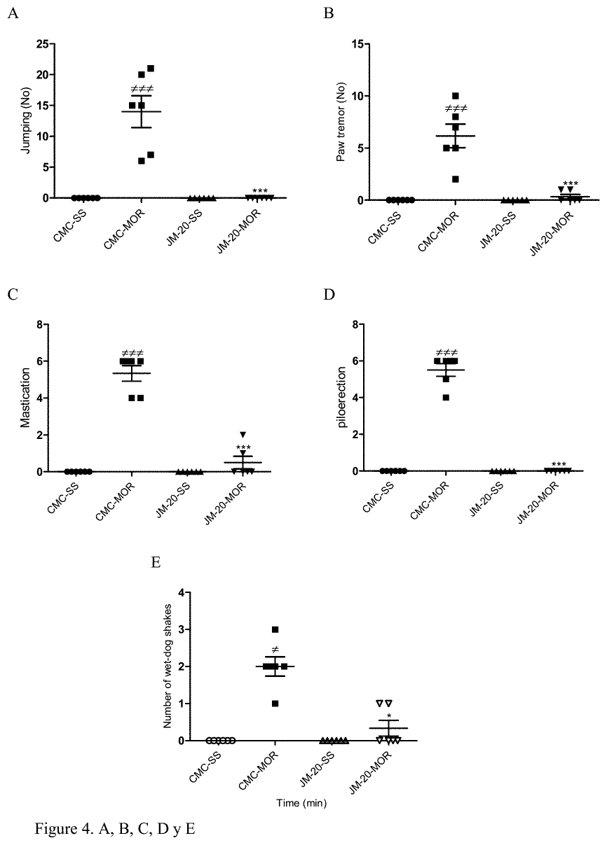

The phenomenon of tolerance expresses the changes that occur in the body as a reaction to chronic or repeated exposure of the same substance and involves pharmacokinetic elements that determine the decrease in the bioavailability of the drug and pharmacodynamic elements (adaptive changes of the opioid system that show the plasticity of the nervous system). Associated with tolerance, physical dependence occurs, both are biological phenomena as a consequence of exposure to the drug and that differ from the concept of abuse and addiction that express a psychological dependence determined by compulsive consumption that involves not only the agent, but also environmental factors and of the host with a genetic, psychological and social dimension (Ballantyne, J C and LaForge, K S 2007. Pain 129: 235-255). As physical dependence results from the state of tolerance or adaptation due to the readjustment of homeostatic mechanisms in the face of repeated administration of the drug, its abrupt suspension produces its imbalance and triggers the search for a new balance in its absence. Consequently, the withdrawal or withdrawal syndrome is a real test of physical dependence (Lu, L. et al. 2000. Eur J Neurosci 12: 4398-4404; Gao, J L et al. 2014. BMC Complementary and Alternative Medicine 14: 308: 1472-688). With continued use of the drug, whether legal or illegal, the phenomena of tolerance and dependence lead to the need to consume the drug to achieve the effect and to alleviate withdrawal symptoms respectively. Consequently, they force drug-seeking behavior (negative reinforcement). In this study from the 3rd day it was observed that the animals treated with morphine showed an ostensible increase in locomotor activity with respect to the other 3 groups. Co-treatment with JM-20 significantly reduced the appearance of spontaneous somatic withdrawal symptoms after withdrawal of morphine, an element that also corroborates that the animals treated with JM-20 are less dependent (FIG. 4. A, B, C, D, E). This would be another benefit of the JM-20-morphine association through the modulation of some pharmacological factors and the agent that facilitate the initiation and maintenance of addiction. In this way, animals that receive a lower dose of morphine in combination therapy and that are less tolerant could be less susceptible to to its establishment. The neurobiology of addiction is based on the discovery of reward centers in the brain, at the level of the mesocorticolimbic system made up of the ventral tegmental area (VTA), the nucleus acumbens (NAc) and the hippocampus, whose final common pathway is dopaminergic. However, they also express opioid receptors extensively, which are the targets for exogenous opioids to cause addiction directly in the NAc and indirectly by decreasing GABAergic inhibition of VTA dopaminergic neurons. The surprising reduction in morphine withdrawal symptoms in animals treated with this BDZ derivative is of particular interest since these drugs also mediate their addictive effects by disinhibiting VTA neurons through interneurons that express GABAA receptors containing the al subunit in this area. Consequently, the expected effect would be a potentiation of hyperactive behaviors induced by morphine, as has been reported for midazolam, for which JM-20 shows an advantage in this regard with respect to the 1,5 known benzodiazepines, probably due to the fusion of a 1,4-dihydropyridine. In this sense, it has been reported that antagonists of type L VGCCs prevent the development of tolerance to opioids and reduce the signs of withdrawal to morphine in rats, as well as the up-regulation of VGCCs sensitive to DHP associated with down-regulation of p receptors in the CNS of tolerant rats to agonists of this opioid receptor (Song L. et al. 2014. Brain Res 20: 1564: 52-61; Michaluk, J. et al. 1998. Eur J Pharmacol 352: 189- 197; Diaz, A. et al. 1995. J. PharmacoL Exp. Ther. 274: 1538-1544). On the other hand, subtype-selectivity and intrinsic activity profile of JM-20 with the different subtypes of a subunits of the GABAA receptor isoforms is unknown. However, this new molecule could have its own characteristics and a lower intrinsic activity on the al subunit, to which the effects of sedation and cognitive worsening are attributed, according to findings in mice with specific mutations of this receptor (Zeilhofer H U et al. 2009 TIPS 30: 97-402). Behavioral studies performed in our laboratory show that JM-20 exerts anxiolytic effects, but does not induce sedation or severe muscle relaxation that affects its performance in the rotarod test. It also shows no signs of tolerance to its analgesic effects at repeated doses or cognitive worsening, but rather has a protective effect on the processes of memory acquisition and storage (Wong-Guerra M. et al. 2019. Neurol Res DOI:10.1080/01616412.2019.1573285).

The withdrawal of the chronically administrated opioid triggers a constellation of symptoms such as anhedonia, hyperalgesia, and noradrenergic symptoms. In this experiment we studied some somatic behaviors and symptoms in the rat associated with spontaneous withdrawal to morphine (jumping or escape attempts, tremors, mastication, piloerection, head shaking). In particular, hypothalamic stress systems through the hypothalamic-pituitary-adrenal (HPA) and extra-hypothalamic [central nucleus of the amygdala (CeA)-locus coeruleus (LC)-nucleus of the solitary tract (NTS)] functionally connected to through the corticotropin releasing factor (CRF) are essential in the neural circuit that regulates this negative state (Laorden, M L et al. 2012. PLoS ONE 7e36871. doi:10.1371/journal.pone.0036871). It has been shown that at the level of the NAc and the LC NMDA receptors are involved in the withdrawal response in rats, the activation of the p receptors increases the post-synaptic NMDA activity in the NAc, in addition there are reports in the clinical setting on the use of NMDA antagonists to reduce the physical and psychological symptoms of opioid withdrawal (Scofield, M D et al. 2016. Pharmacol Rev 68: 816-871). The release of glutamate in the LC precipitated by withdrawal is regulated by L-type Ca²⁺ channels and is involved in these symptoms, which can be inhibited by blockers of this channel such as diltiazem (Tokuyama, S. et al. 1995. Eur J Pharmacol 279: 93-98). In general, the dual action of opioid receptors and L-type Ca²⁺ channels by increasing the responses mediated by the NMDA receptor and facilitating the release of glutamate, respectively, provides another mechanism to explain the control of CNS excitability that these substances exert. Both targets are frequently activated simultaneously by synaptic signals and could interact synergistically to increase excitatory conduction and intracellular biochemical signaling controlled by Ca²⁺ concentrations. In particular, oscillations of this ion regulate gene expression and second messenger activity with an impact on the long-term effects of opioids on synaptic plasticity and genomic regulation. In our laboratory it has been possible to observe the persistence of the long-term preventive effect of JM-20 on the development of tolerance/hyperalgesia, 12 days after its discontinuation in CCI animals previously treated with morphine for 4 days (CCI-MOR-CMC=44.82±1.39 vs. CCI-MOR-JM-20=16.40±3.8) (p<0.001) (Table 4). Likewise, the contribution of glia-neuron signaling in the periaqueductal gray matter (PAG) to opioid dependence and withdrawal syndrome has been reported. PAG is located in the middle of this circuit and the release of TNF-a by activated microglia via the p-TLR4-NFKB receptor has been identified as a new therapeutic target. Its interaction with the TNFR1 receptor can induce phosphorylation of ERK, CREB and NMDA on PAG neurons, causing transcriptional changes and synaptic plasticity (Ouyang, H. et al. 2012. The Scientific World J. doi:10.1100/2012/940613). A plausible elucidation to explain the reduction of these excitatory and hyperactive behaviors in animals co-treated by JM-20 presumes the participation of the fraction with L-type Ca²⁺ channel antagonist activity in the structure of this multimodal molecule, which also is a modulator of glutamatergic signaling and neuroinflammation as reported in ischemia models (Núñez-Figueredo, Y. et al. 2015. Neurochem Int 81: 41-7; Ramirez-Sánchez, J. et al. 2015; Neurochem Int 90: 215-223; Ramirez-Sánchez, J. et al. 2018. Mol Neurobiol https://doi.org/10.1007/s12035-018-1087-8).

TABLE 1
General behavioral evaluation of the nervous system day 1

| | Rota-rod | | | | |
|---|---|---|---|---|---|
| | motor | Placing/ | | | |
| | performance | stepping | Righting | Pinna | Corneal |
| | (Latency sec) | reflex | reflex | reflex | reflex |
| CMC-SS | 58 ± 1.8 | 100 | 100 | 100 | 100 |
| CMC-MOR | 16 ± 1.9^{a} | 100 | 100 | 100 | 100 |
| JM-20-SS | 59 ± 0.5^{b} | 100 | 100 | 100 | 100 |
| JM-20-MOR | 32 ± 5.4^{a,b} | 100 | 100 | 100 | 100 |

Data are expressed as mean ± SEM of performance, cut-off was 60 minutes at 22 rpm and % of animals showing each of the reflexes.
Measurements carried out 5 minutes prior to the evaluation of nociceptive responses, 45 minutes after the first dose of morphine or saline and 105 minutes after the administration of JM-20 (20 mg/kg, p.o.) or CMC 0.05%.
n = 6 per group.
^{a}p ≤ 0.001 vs CMC-SS group,
^{b}p ≤ 0.001 vs CMC-MOR group, one-way ANOVA followed by Bonferroni's test

TABLE 2
General behavioral evaluation of the nervous system day 5

| | Rota-rod | | | | |
|---|---|---|---|---|---|
| | motor | Placing/ | | | |
| | performance | stepping | Righting | Pinna | Corneal |
| | (Latency sec) | reflex | reflex | reflex | reflex |
| CMC-SS | 60 | 100 | 100 | 100 | 100 |
| CMC-MOR | 57 ± 1.8 | 100 | 100 | 100 | 100 |
| JM-20-SS | 59 ± 0.5 | 100 | 100 | 100 | 100 |
| JM-20-MOR | 52 ± 1.5 | 100 | 100 | 100 | 100 |

Data are expressed as mean ± SEM of performance, cut-off was 60 minutes at 22 rpm and % of animals showing each of the reflexes.
Measurements carried out 5 minutes prior to the evaluation of nociceptive responses, 45 minutes after the morphine or saline dose, and 105 minutes after the administration of JM-20 (20 mg/kg, p.o.) or CMC 0.05%.
n = 6 per group, NS between groups.
One-way ANOVA followed by Bonferroni's test

TABLE 3
General behavioral evaluation of the nervous system day 11

| | Rota-rod | | | | |
|---|---|---|---|---|---|
| | motor | Placing/ | | | |
| | performance | stepping | Righting | Pinna | Corneal |
| | (Latency sec) | reflex | reflex | reflex | reflex |
| CMC-SS | 60 | 100 | 100 | 100 | 100 |
| CMC-MOR | 60 | 100 | 100 | 100 | 100 |
| JM-20-SS | 60 | 100 | 100 | 100 | 100 |
| JM-20-MOR | 54 ± 1.8 | 100 | 100 | 100 | 100 |

Data are expressed as mean ± SEM of performance, cut-off was 60 minutes at 22 rpm and % of animals showing each of the reflexes.
Measurements carried out 5 minutes prior to the evaluation of nociceptive responses without medications, n = 6 per group, NS between groups.
One-way ANOVA followed by Bonferroni's test

TABLE 4
Effect of pre-treatment with JM-20 on the intensity of mechanical hypernociception in
mononeuropathic rats after chronic administration of morphine or saline

| | SS | | | MOR | | |
|---|---|---|---|---|---|---|
| | 7 | 11 | 23 | 7 | 11 | 23 |
| CCI | 41.41 ± 1.45 | 41.48 ± 1.74 | 43.12 ± 1.53 | 41.22 ± 1.58 | 45.09 ± 1.60* | 44.82 ± 1.39* |
| Sham | 1.16 ± 1.48≠ | 5.37 ± 1.89≠ | 7.39 ± 0.88≠ | 2.06 ± 2.96≠ | 40.49 ± 2.57* | 37.49 ± 2.10* |
| CCI | | | | | | |
| JM-20 | 41.55 ± 1.50 | 10.68 ± 5.89*≠ | 5.93 ± 2.65*≠ | 41.82 ± 1.2.03 | 17.18 ± 3.35*≠ | 16.40 ± 3.82*≠ |
| CCI | | | | | | |

Starting on day 7 post-surgery, morphine 10 mg/kg or saline (SS) was administered s/c every 12 hours for 4 days and in the group pre-treated with JM-20 (10 mg/kg, p.o.), it was administered at single dose for 5 days. The intensity of hypernociception was measured on days 7, 11 and 23 post-surgery, in correspondence with the times prior to the start of treatments, 6 hours after 5 days of the administration of JM-20 and 19 hours after the last dose of morphine and finally 12 days after the discontinuation of JM-20 respectively. The data are presented as mean ± SEM of the difference (Δ) of the withdrawal threshold in grams calculated from the subtraction of the mean of the three measurements at the different time intervals from the mean of the three measurements at time 0, n = 6 per group of animals
*p ≤ 0.05 compared to values at day 7,
≠p ≤ 0.001 compared to CCI rats. (T test to compare two means, for 3 or more means one-way ANOVA followed by Dunnett's test).

The design in the CCI model pursues the evaluation of the possible preventive ability of JM-20 on the induction of these neuroadaptations in the context of neural injury, in which the facilitation of the development of tolerance to analgesia by morphine has been reported (Bennett, G J and Xie, Y K 1988. Pain 33: 87-107; Christensen, D. and Kayser, V. 2000. Pain 88: 23-238). In baseline conditions, the withdrawal threshold value of the right hind paw when faced with a mechanical stimulus was 46.75±0.9 g (mean±SEM, n=36). Subsequently, the intensity of mechanical hypernociception was tested using the (Δ) of the withdrawal threshold in grams with respect to the baseline values at 7 days' post-surgery, when the administration of morphine 10 mg/kg or SS was started subcutaneously every 12 hours for 4 days and JM-20 or its vehicle orally (Cunha, T. et al. 2004. Braz. J. Med. Biol. Res. 37: 401-407). In this way, the following 6 experimental groups were conformed (n=6 animals each): sham CCI group treated with saline (Sham-SS), sham CCI group treated with morphine (Sham-MOR), CCI group treated with saline (CCI-SS), CCI group treated with morphine (CCI-MOR), CCI group treated with saline s.c. and JM-20 p.o. (CCI-SS-JM-20) and CCI group treated with morphine s.c. and JM-20 p.o. (CCI-MOR-JM-20). The mechanical thresholds were evaluated again 5 days after starting the oral treatment of JM-20 (10 mg/Kg, p.o.) or CMC 0.5%, a moment that coincides with day 11 post-surgery. From that moment on, all treatments were discontinued for 12 days, a time that coincides with day 23 post-surgery and finally the withdrawal thresholds to mechanical stimulation were evaluated in order to explore the maintenance of neuroadaptations and the impact of the treatment with JM-20 in the long term. The animals were sacrificed, after taking a sample of the sciatic nerve to study any possible impact on the Wallerian degeneration (WD)-related histopathological changes (Debový, P. 2011. Annals of Anatomy 193: 267-275). As can be seen in Table 4, CCI animals showed an increase in the intensity of mechanical hypernociception with respect to the sham ones operated as a control for the model at day 7 post-surgery, in addition there were no differences in the intensity of hypernociception between the groups of mononeuropathic rats at this point the different treatments are started. Co-treatment with JM-20 for 5 days increased mechanical withdrawal thresholds and consequently reduced the intensity of hypernociception in CCI group that received SS s.c. with respect to its control group treated with vehicle at day 11 post-CCI. Measurement was performed 6 h after the last dose of JM-20 (CCI-SS=41.48±1.74 vs. CCI-SS-JM-20=10.68±5.89) (p <0.001). This effect was maintained in the long term 12 days after suspension of treatment 23 days post-CCI (CCI-SS=43.12±1.53 vs. CCI-SS-JM-20=5.93±2.65) (p<0.001). This suggests its ability to prevent synaptic plasticity changes induced by neural injury and is in agreement with our previous findings associated with the decrease in histopathological changes of WD. On the other hand, the CCI animals treated with morphine showed evolutionarily greater intensity of hypernociception with values of 45.09±1.60 at day 11 (p<0.05) and of 44.82±1.39 at day 23 (p<0.05) with respect to its intensity at day 7 post-surgery when they begin to take morphine (41.22±1.58), as was also observed in the sham operated animals (Table 4). Even under these conditions, JM-20 significantly reduced the intensity of mechanical hypernociception compared to its vehicle-treated CCI-MOR control animals at day 11 post-CCI (CCI-MOR-CMC 45.09±1.60 vs CCI-MOR-JM-20 17.18±3.35) (p<0.001). The long-term effect of the product was also observed after the administration of repeated doses of morphine, which suggests its preventive capacity to mitigate the changes in synaptic plasticity in the face of chronic exposure to morphine. As explained in the background, many of the mechanisms that mediate these changes in both paradigms are similar (CCI-MOR-CMC=45.18±1.5 vs CCI-MOR-JM-20=16.40±3.82) (p<0.001) (Mayer, D J et al. 1999. Proc Natl Acad Sci USA 96: 7731-7736).

The preservation of the response to an acute dose of morphine intraperitoneally (1 mg/kg i.p.) was also evaluated in another group of CCI animals exposed or not to repeated doses of morphine 10 mg/kg or SS s.c. every 12 hours for 4 days previously, as well as the possible influence of the pretreatment with JM-20 on it. The experiment was carried out 11 days after surgery and at 19 hours after the last s.c. injection of the previous day. After evaluating the control withdrawal thresholds, the intensity of mechanical hypernociception (Δ of withdrawal threshold in grams with respect to baseline values) was determined prior to acute morphine injection (time 0).

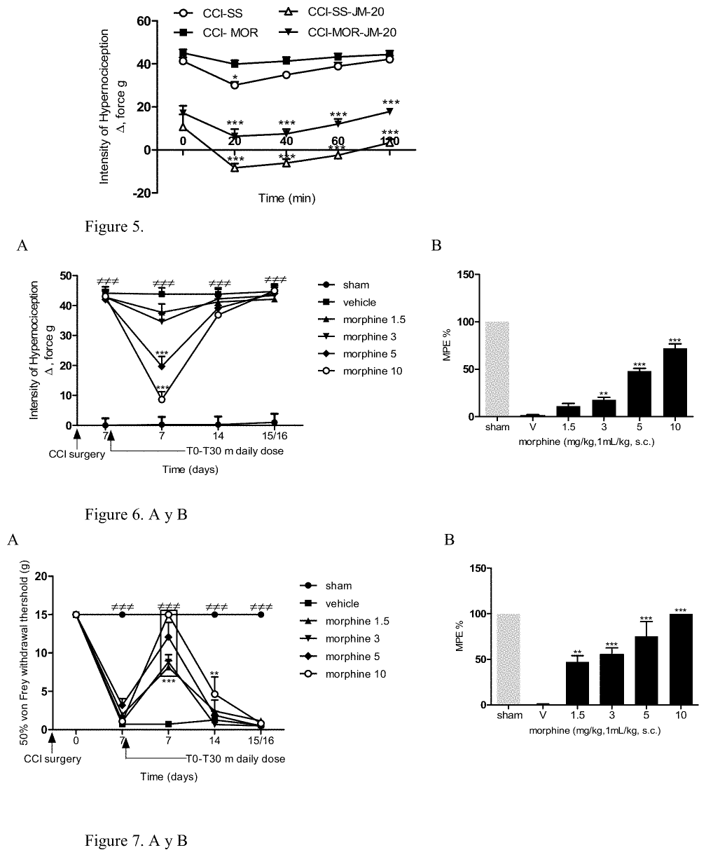

Posteriorly, the evaluations were continued at 20, 40, 60 and 120 minutes after its administration, until the threshold values returned to their controls (intensity of hypernociception at time 0). The CCI-SS group, despite neural injury and its high intensity of hypernociception, significantly preserved its response to morphine at 20 minutes after its administration by this route (41.29±1.74 vs 30.13±1.55, p<0.05) which was not observed in the CCI-MOR group (45.09±1.60 vs 39.92±1.78, FIG. 5). Animals previously exposed to repeated doses of morphine showed a reduction in the intensity and duration of its acute effect, in correspondence with that reported by other authors. It has previously been shown that, after a pre-treatment regimen with morphine, its acute administration retains its activity against noxious mechanical and thermal stimuli in non-operated rats, but is significantly reduced in CCI rats and with a tendency to decrease in sham operated animals. (Christensen, D. and Kayser, V. 2000. Pain 88: 23-238). However, the CCI-SS-JM-20 animals that included JM-20 in their pre-treatment regimens for 5 days significantly reduced the intensity of mechanical hypernociception at the acute dose of morphine at 4 measurement times.

That is, its effect exceeded 20 minutes and was maintained until 120 minutes (10.68±5.89 vs. -8.33±2.03; -6.11±1.90; -2.45±1, 13, p<0.001; 3.34±1.71, p<0.05, FIG. 5) respectively, which suggests a reinforcement of the opioid effect, possibly due to synergistic and/or additive mechanisms that should be explored through a study of drug interaction. On the other hand, in the CCI-MOR-JM-20 group, the conservation of the response to its acute dose was observed up to 40 minutes (17.18±3.35 vs 6.40±3.28; 7.52±2.19, p<0.01, FIG. 5), suggesting a protective effect of JM-20 on the induction of tolerance to morphine under conditions of neural injury and consequently improving its efficacy (FIG. 5). For individual comparisons, the Bonferroni's test (one-way ANOVA) was used. 2-way analysis of repeated measures ANOVA to determine differences between groups after treatment with the acute dose of morphine, revealed significant main effects of time (F4.80=36.32, p<0.0001) and treatment (F3, 80=125.14, p<0.0001) and a significant interaction between time x treatment (F12.80=2.76, p=0.0034).

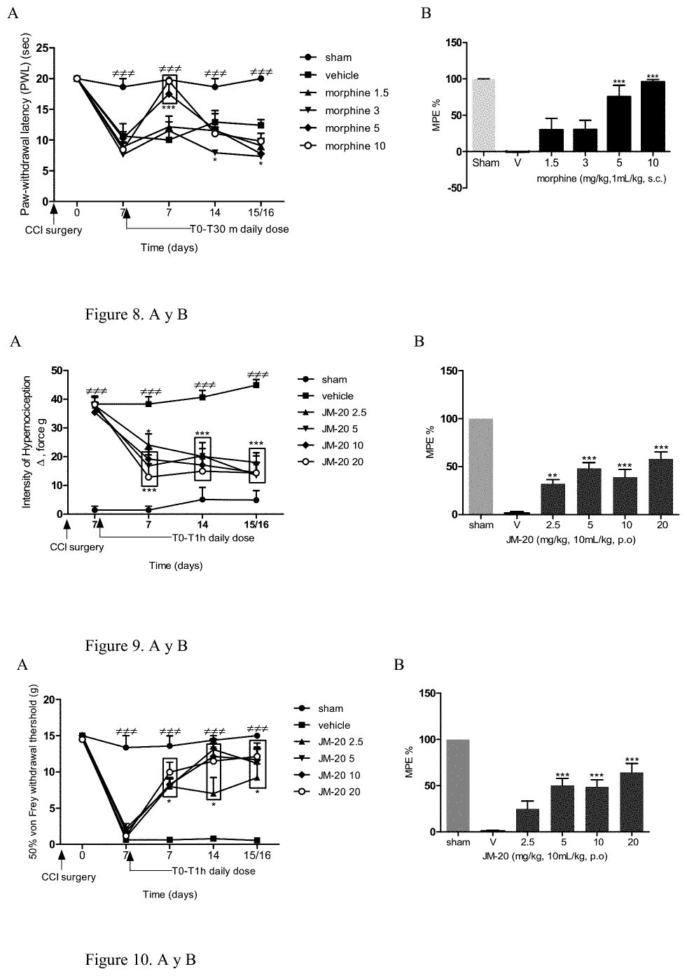

In order to study the dose-response curves of morphine and compound JM-20 by subcutaneous and oral routes respectively, other experiments were designed in CCI model to establish the effective dose ED30 or ED50 according to % of maximum possible effect (MPE) produced by each drug to reduce each of the sensory symptoms of neuropathic pain reproduced by the model (Jensen, T S and Baron, R. 2003. Pain 102: 1-8). In this way, the study of drug interaction in a realistic way, not only at a single dose, as is usually reported in isobolographic studies, but also to corroborate the efficacy of the combination product also at repeated doses, in this case to avoid the establishment of tolerance/hyperalgesia to morphine in these conditions (Moreno-Rocha, L A et al., 2012. Pharmacol Biochem Behav 103: 1-5). Therefore, the design included the evaluation of increasing doses of morphine repeatedly for 8 days, mimicking the context clinical trial of the treatment of patients with neuropathic pain and the procedure was similar for the JM-20. Once the model had been reproduced, the withdrawal threshold of the injured hind paw was measured upon stimulation with von Frey filaments and upon stimulation with an electronic von Frey, as well as upon thermal stimulation using the Hargreaves test, to determine the presence of mechanical allodynia, mechanical hypernociception and thermal hypernociception respectively. The withdrawal thresholds after the treatments were converted into % MPE [mechanical allodynia: MPE% =(post-treatment threshold-pre-treatment threshold)/(15g-pre-treatment threshold)×100; Mechanical hypernociception: MPE %=(Δg post-treatment−Δg pre-treatment)/50 g−Δg pre-treatment)×100; MPE %=(PWL post-treatment−PWL pre-treatment)/20 sec-PWL pre-treatment)×100]. Measurements were carried out under baseline conditions and 7 days post-CCI (peak of hyperalgesia in the model), at which time the experimental groups (n=6-7 per group) were formed to start the studies. The morphine doses were selected from previous reports and those of JM-20 from the studies carried out in our laboratory in this model, in which the primary role of neuroinflammation within its pathophysiological mechanisms is recognized (Berger, J. V. et al. 2011. Brain Res Rev 267: 282-310). This is an element that supports the better anti-allodynic response to the single dose of morphine in this scenario despite the known resistance to opioids in general in neuropathic pain and consequently its low efficacy in models of painful post-traumatic neuropathy (De Vry, J et al., 2004. Eur J Pharmacol 491: 137-148). The ipsilateral paw withdrawal response was evaluated again at day 14 post-CCI, 1 h after the last administration and at day 15 post-CCI, 1 day after the withdrawal of morphine. This last measurement is used to evaluate hyperalgesia induced by opioid withdrawal. Fixed-dose combinations of two or more drugs of different classes (FDCs) are an alternative within the types of pharmacotherapeutic combinations that are currently used. However, combinations of drugs from the same class that differ in their pharmacokinetics (i.e. immediate and extended release) and combinations of drugs released from different routes of administration (i.e. topical agent and oral agent) are also available (Mao, J. et al., 2011. J Pain 12: 157-166). Morphine administered subcutaneously begins its action in 10 to 15 minutes, this is maximum in 60 to 90 minutes and lasts between 4 to 6 hours, its most sustained effect by this route makes it the choice in the palliative care of cancer patients. With respect to compound JM-20, we have observed transient biological and particularly antihypernociceptive activity between 30 minutes to 1 hour, maintained up to 3 hours after its oral administration in a single dose. In addition, its effects are maintained after discontinuation of treatment (long-term effect), hence the proposal shows not only the pharmacodynamic rationale suggested in previous paragraphs, but also pharmacokinetics to improve the efficacy of morphine in neural injury conditions (Gilron, I. et al., 2013. Lancet Neuro/12: 1084-1095). The following 6 experimental groups are formed: Sham CCI group, CCI group treated with vehicle (SS 1 mL/kg, s.c.) and CCI groups treated with morphine 1.5, 3, 5 and 10 mg/kg, s.c.). FIG. 6A shows the mechanical antihypernociceptive effect expressed as intensity of hypernociception (Δ force, g) of increasing doses of morphine at 30 minutes after its first administration 7 days post-CCI. This was significant for the doses of 5 and 10 mg/kg vs. vehicle (p <0.001). The decrease in its effect (tolerance) is also observed after the administration of 8 repeated doses at 14 days post-CCI and the hyperalgesia induced after its withdrawal evaluated on days 15-16 post-CCI. For the intensity of mechanical hypernociception, morphine according to the doses used, showed the following MPE values: 1.5 mg/kg=11.23±2.6%, 3 mg/kg=17.67±2.8% (p<0.01), 5 mg/kg=48.05±2.9% (p<0.001), 10 mg/kg=72.22±4.6% (p<0.001), ED50=5.63±0.8 (FIG. 6B). FIG. 7A shows the mechanical anti-allodynic effect of increasing doses of subcutaneous morphine expressed as a 50% withdrawal threshold to the von Frey filaments after their first administration. The effect is significant for all doses 1.5-10 mg/kg (p<0.001) vs. vehicle, however, tolerance to this effect is established after repeated doses and it disappears completely by days 15-16 post-CCI after withdrawal. For the mechanical allodynia, morphine according to the doses used, showed the following MPE values: 1.5 mg/kg=47.45±6.6% (p<0.01), 3 mg/kg=55.28±7, 5% (p<0.001), 5 mg/kg=75.30±16.2% (p<0.001), 10 mg/kg=100%±0.0 (p<0.001), ED50=1.95±0, 3 (FIG. 7B). FIG. 8A shows the dose-dependent thermal antihypernociceptive effect of morphine expressed as paw withdrawal latency, this was significant for the doses of 5 and 10 mg/kg vs. vehicle ((p<0.001). The establishment of morphine induced-neuroadaptations after 8 repeated daily doses was also found for this sensory alteration. For thermal hypernociception, morphine according to the doses used, showed the following MPE values: 1.5 mg/kg=29.69±16.1%, 3 mg/kg=30.0±13%, 5 mg/kg=75.32±15.9% (p<0.001), 10 mg/kg=95.69±3.4% (p<0.001), ED50=3.17±0.4 (FIG. 8B). In a similar way, the dose-response curve of JM-20 was constructed, the following 6 experimental groups were conformed: sham CCI group, CCI group treated with vehicle (CMC 0.05%, 10 mUkg, p.o.) and CCI groups treated with JM-20 (2.5, 5, 10 and 20 mg/kg, p.o.). At day 7 post-CCI, the first administration of the drug is started and 60 minutes after its single oral dose, measurements are performed. FIG. 9A shows the acute mechanical antihypernociceptive effect of JM-20, this was significant with respect to the animals treated with its vehicle from doses of 2.5 mg (p<0.05), better at doses of 5-20 mg/kg (p<0.001), however, no major differences were observed between the doses. This effect is maintained on days 14, 15-16 post-CCI after repeated doses of this compound for 8 days, thus, neuroadaptations (tolerance/hyperalgesia) were not observed after repeated administration. For the intensity of mechanical hypernociception, JM-20 according to the doses used, showed the following MPE values: 2.5 mg/kg=32.26±4.2% (p<0.01), 5 mg/kg=48.02±6.3% (p<0.001), 10 mg/kg=39.20±7.9% (p<0.001), 20 mg/kg=58.15±7.3% (p<0.001), ED50=12.41±6.4 (FIG. 9B). FIG. 10A shows the mechanical anti-allodynic effect of the increasing doses of JM-20 expressed as 50% withdrawal threshold to von Frey filaments 1 h after their first administration, this was significant with respect to the animals treated with its vehicle from doses 2.5 mg/kg, better at doses of 5-20 mg/kg (p<0.05). In this modality, no large differences were observed between the doses used and its effect is maintained at 14, 15-16 days post-CCI after its administration for 8 days, that is, no neuroadaptations (tolerance/hyperalgesia) were induced after the repeated doses of this compound. For mechanical allodynia, JM-20 according to the doses used, showed the following MPE values: 2.5 mg/kg=24.97±8.5%, 5 mg/kg=50.21±7.6% (p<0.001), 10 mg/kg=48.68±7.6% (p<0.001), 20 mg/kg=64.25±9.7% (p<0.001), ED50=8.44±1.9 (FIG. 10B). In correspondence, with other experiments, JM-20 did not increase withdrawal thresholds significantly in the Hargreaves test after its first administration or at repeated doses at 14 days post-CCI (FIG. 11A and B). However, a moderate non-dose-dependent increase was observed in the animals treated with JM-20 at 15-16 days post-CCI with respect to the control animals (vehicle) (FIG. 11A). In order to study the possible synergistic effect of this pharmacotherapeutic combination and its efficacy in preventing long-term morphine-induced neuroadaptations, the theoretical ED was calculated from the ED50 values at a single dose of both compounds in the modality mechanical hypernociception. In this way, we hypothesized that by reducing the morphine doses in the combination and due to the neuroprotective mechanisms of the JM-20 compound, better MPE % could be observed at 14 and 15/16 days post-CCI compared to those observed with the morphine doses alone. The results on this variable were selected, given the susceptibility of the afferent mechanical systems to hyperalgesia associated with repeated injections of morphine, corroborated in the experiments (FIG. 6A and B). Likewise, these systems could be an interesting target for the JM-20, as suggested by our previous findings, which have shown its preference for action over mechanical systems and the absence of activity in thermal nociception examinations such as hot plate and Hargreaves tests, in which morphine has a potent effect (Christensen, D. and Kayser, V. 2000. Pain 88: 23-238). Theoretical ED (EDt) was 9.02 ±3.3 mg/kg and the calculated doses to be evaluated were 0.6, 1.1, 2.3, 4.5 and 9 mg/kg at proportions of 0.3 for morphine and 0.7 for JM-20. After 7 days of induction of damage by CCI to the animals and their evaluation at time 0, the same experimental groups were conformed according to the designs used for the independent drugs and the daily treatments were started for 8 days with the combinations: C1=1.1 mg/kg, C2=2.3 mg/kg, C3=4.5 mg/kg, C4=9 mg/kg.

TABLE 5
Antihypernociceptive doses (mg/kg) used in the study of the
interaction between morphine and compound JM-20 after their
administration by subcutaneous and oral routes respectively,
in the sciatic chronic constriction injury model

| Morphine in the | JM-20 in the | Total dose in the |
|---|---|---|
| combination | combination | combination |
| 0.33 | 0.77 | 1.1 |
| 0.69 | 1.61 | 2.3 |
| 1.35 | 3.15 | 4.5 |
| 2.70 | 6.30 | 9.0 |

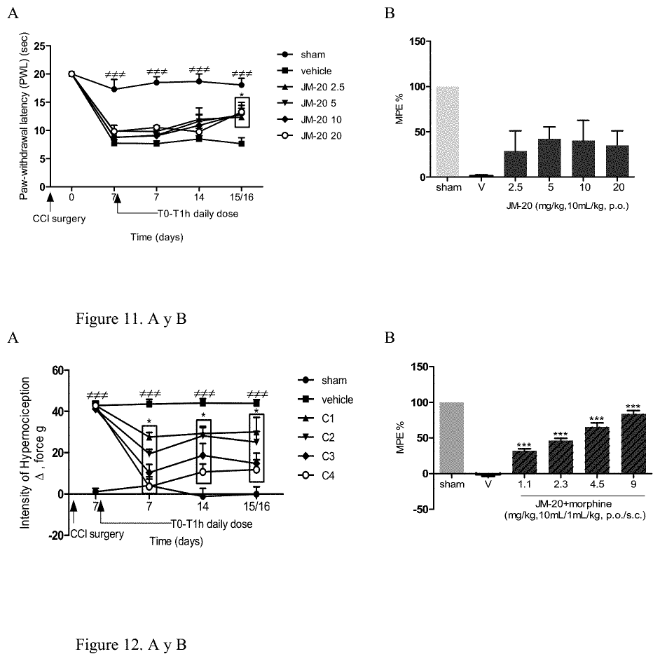

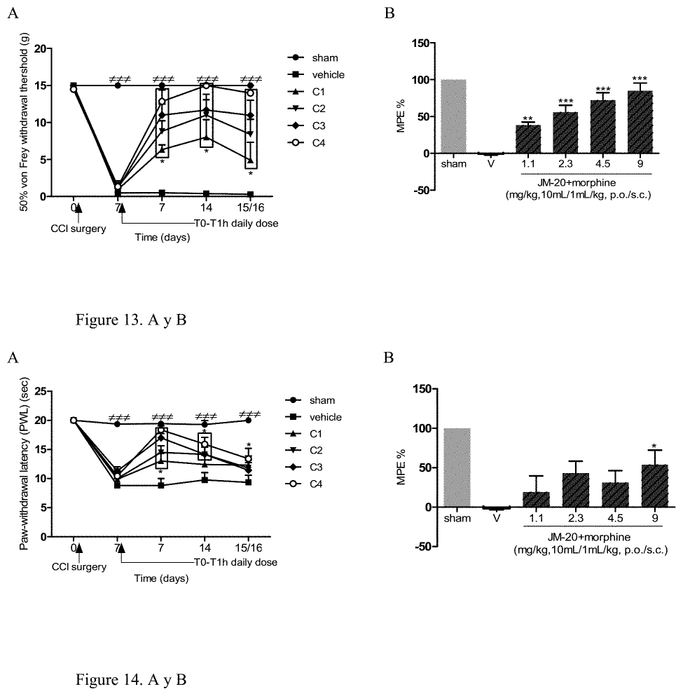

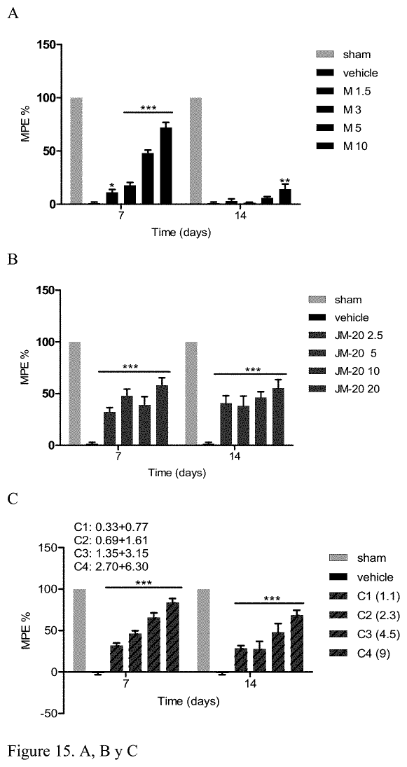

The mechanical anti-hypernociceptive and mechanical anti-allodynic effects of the C1-04 combinations in a dose-dependent manner was observed (p<0.05) at 7 days post-CCI, effects that were maintained at 14 days after 8 repeated doses and at 15-16 days post-CCI after its discontinuation (FIG. 12A and FIG. 13A). The combinations C1-C4 were also effective in increasing the withdrawal thresholds in the Hargreaves test in a dose-dependent manner (p<0.05) at 7 days post-CCI and their effects were maintained for C2-C4 (p<0.05) at 14 days and for C3-C4 at 15/16 days post-CCI (FIG. 14A). In FIGS. 12 B, 13 B and 14 B, the therapeutic effect of the single dose of these combinations is expressed as MPE% for the 3 sensory alterations respectively. In a translational paradigm for 8 repeated doses of the morphine-JM-20 combination by these two routes, it was observed that, although the % of MPE evaluated at day 14 decreases, the therapeutic effect is maintained significantly and above 50% for the combination C4. This result constitutes an evidence of the preventive effect of these combination on morphine induced-tolerance, unlike the animals treated with morphine alone in which the reduction of its efficacy is observed (FIG. 15A, B, C).

% MPE morphine (M) s.c. at 7 days post-CCI: M1.5=11.22±2.6%; M3=17.67±2.8% (p<0.01); M5=48.05±2.9% (p<0.001); M10=72.22±4.6% (p<0.001) vs. at MPE 14 days post-CCI: M1.5=2.96±2.0%; M3=1.36±0.5%; M5=6.10±1.0%; M10=14.18±4.7%% (p<0.01).

% MPE JM-20 p.o. 7 days post-CCI: JM-20 2.5=32.26±4.2% (p<0.01); JM-20 5=48.02±6.3% (p<0.001); JM-20 10=39.20±7.9% (p<0.001); JM-20 20=58.15±7.3% (p<0.001) vs. % MPE 14 days post-CCI: JM-20 2.5=40.86±7.1% (p<0.001); JM-20 5=38.13±9.4% (p<0.001); JM-20 10=46.18±5.7% (p<0.001); JM-20 20=55.28±8.3% (p<0.001).

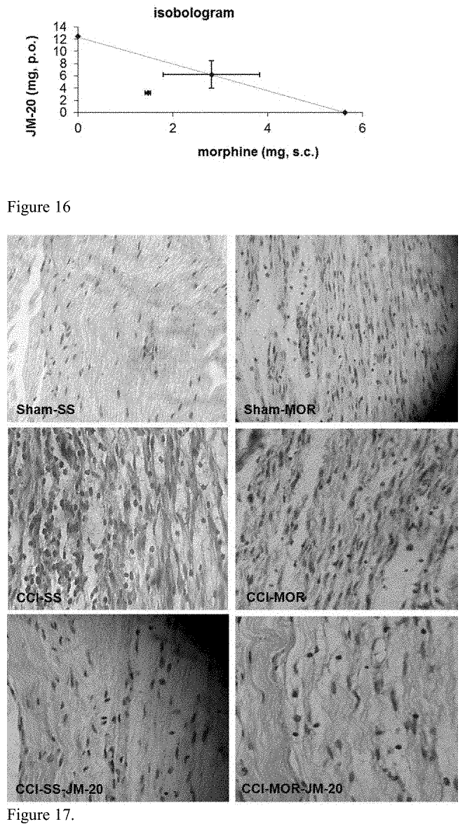

% MPE morphine s.c.-JM-20 p.o. combination 7 days post-CCI: C1=32.03±2.9%; C2=46.49±3.2; C3=65.67±5.6%; C4=83.93±4.7% vs.% MPE 14 days post-CCI: 01=28.52±3.2; C2=27.94±8.9; C3=47.88±10.5; C4=68.88±5.7. The most effective combination was C4: 9 mg/kg (morphine 2.70 mg/kg, s.c. and JM-20 6.30 mg/kg, p.o.). The method makes it possible to reduce the doses of both compounds and particularly by reducing the doses of morphine, tolerance/hyperalgesia induced by its repeated doses, more frequently observed at high doses, is prevented, improving its efficacy in conditions of neural injury. There were no signs of CNS depression. The experimental ED (EDe) was 2.41±0.08 mg/kg, then the combination acted supra-additively to reduce the mechanical hypernociception evaluated one hour after its first administration at 7 days post-CCI. The value of the interaction index was 0.267±0.09 and the absolute value of t'3.63 was greater than that of T 1.81, demonstrating its statistical significance p<0.05 (Student-T). (FIG. 16). The administration of repeated doses of this fixed-dose combination showed improved therapeutic efficacy than morphine alone in reducing mechanical hypernociception in mononeuropathic rats. The pharmacological interaction and safety studies provide elements of the higher pharmacological profile of our proposal, such as the reduction of the dosage of both drugs and adverse effects of acute installation (sedation and respiratory depression), its supra-additive analgesic effect not only immediate, but in the long term by increasing the analgesic efficacy of the opioid in a neuropathic pain model.

Although the focus of attention to explain the mechanisms of these neuroadaptations is directed towards the CNS, with a primary role of the spinal mechanisms, the contribution of the peripheral nervous system to OIH has now begun to be studied. Consequently, it was decided to characterize some histopathological manifestation under the light microscope of the effect of the chronic administration of morphine in sham operated animals with neural injury. Mononeuropathic animals (CCI-SS) show increased relative cellularity with respect to sham operated animals as a result of Schwann cell (SC) proliferation and macrophage infiltration. The presence of digestion chambers in SC cells with myelin ovoids and disorderly alignment of axons with loss of their myelin sheaths, indicative characteristics of Wallerian degeneration, are observed. While the sham operated animals show an orderly alignment of their axons that preserve their myelin sheaths, as well as a low cellularity. Qualitatively, as observed in FIG. 17, the animals pre-treated with morphine are compared with those pre-treated with saline solution in these same paradigms of real CCI surgery and sham surgery. Interestingly, it was observed that the animals of the Sham-MOR group predominantly showed a minimal increase in cellularity compared to the animals of the Sham-SS group, as well as perivascular infiltrate of lymphocytes. In addition, proliferation of SC and mild vascular congestion were observed, as evidence of a peripheral neuroinflammatory response to the repeated administration of morphine, even in the absence of physical neural injury. The presence of proliferation, although minimal, of these cells in Sham-MOR animals, as well as perivascular cellular infiltration, unlike those treated with SS, could partly explain the hypernociception observed in these animals. On the other hand, MOR-CCI animals showed similar signs of axonal degeneration than SS-CCI, but with higher and more variable cellularity, including the presence of Langhans cells and significantly less presence of digestion chambers with myelin ovoids in SCs than the SS-CCI, which suggests the reduction of clearance of myelin debris during the first week after the injury in these animals pre-treated with morphine and consequently the perpetuation of the neuroinflammatory process through the recruitment of macrophages, the production of pro-inflammatory cytokines and hypernociception. The ineffective clearance of myelin debris after neural damage is also the factor that most limits regeneration and is the cause of chronic neuropathic pain. It has been suggested that deficiency of the phagocytosis and autophagy processes of SCs may be an early event that facilitates the initiation of neuropathic pain. SC initiates and collaborates with macrophages in this clearance through phagocytosis through TAM receptors and autophagy, its role at 5 days after CCI is essential to prevent the onset of neuropathic pain and facilitate regeneration in this model. Inhibitors of the mammalian cell target for rapamycin (mTOR) are inducers of autophagy and may promote analgesia. In particular, increased expression of mTOR in the SDH after repeated intrathecal morphine delivery has been implicated in OIH (Rivat, C. and Ballantyne, J. 2016. Pain Reports 1: e570; Wei, Z et al. 2019. Front. Cell. Neurosci. 13: 116). Faced with a greater neuroinflammatory challenge imposed by continued treatment with morphine in CCI-MOR animals, co-treatment with JM-20 (CCI-MOR-JM-20) still exerts its neuroprotective effect. These animals presented moderate to marked degeneration with few areas of conserved nerve fibers, decreased vascular congestion and cellularity compared to CCI-MOR animals co-treated with vehicle. However, when comparing them with the CCI-SS-JM-20 group, it is observed that the preservation and/or axonal regeneration in them is greater in correspondence with a lower neuroinflammatory process. The histopathological variables are in correspondence with the sensory manifestations and the potency of the effect of the acute dose of morphine in the animals co-treated with JM-20 in both groups of CCI animals, superior in the animals not chronically exposed to morphine (FIG. 5). Although the administration of JM-20 was started 7 days after the induction of neural injury once hyperalgesia was established, the WD processes that start 24 hours after the injury continue for 3 or more weeks, therefore that a preventive effect in early stages on the evolution of these processes could be significant. In particular, the invasion by neutrophils of the endoneurium distal to the lesion that occurs around 7 days, contributes to the peripheral mechanisms of neuropathic pain (Ramer, M S et al. 1997. Pain 72: 71-8; Berger, J V et al. 2011. Brain Res. Rev. 67: 282-310; Debový, P. 2011. Annals of Anatomy 193: 267-275). JM-20 inhibits neutrophil migration events during the inflammatory process, as well as the release of TNFα, which is essential for macrophage recruitment and in the initiation of the neural degenerative process (Tai, Y H et al. 2006. Pain 124: 77-86; Arsenault, A. and Sawynok, J. 2009. Pain 146: 308-314; Üçeiler, N. and Sommer, C. 2006. Drug Discovery Today: Disease Mechanism. DOI:10.1016/j.ddmec.2006.09.004; Nagano, M. et al. 2003. Br. J. Pharmacol. 140: 1252-60; Griffin, J W 2006. In: Campbell, J N, Basbaum, A I, Dray, A., Dubner, R., Dworkin, R H, Sang, C N, editors. Emerging strategies for the treatment of neuropathic pain. Seattle: IASP Press. P. 271-90; Austin, P L and Moalem-Taylor, G. 2010. Journal of Neuroimmunology 229: 26-50).

### EXAMPLES OF REALIZATION

### Example 1

### Evaluation of Nociceptive Behaviors in Tolerant/Hyperalgesic Rats

Measurement of allodynia and mechanical hypernociception The animal must have its four paws supported, when it is placed in the test boxes (inverted plastic boxes with 21×16×27cm³ lids) that rest on a metal or glass mesh floor according to the test) it cannot be exploring or grooming at the time of measurement, the adaptation period of 5-10 minutes was completed. In the first case, the withdrawal response to the filaments of the two hind paws was counted as % response to exposure to the corresponding filament. Filaments 4, 8 and 15 g were applied in an ascending manner on the middle plantar area on both hind paws, each one 5 times for 5 seconds (total of 10 exposures), after their confinement and adaptation to their observation boxes for 10 minutes. The response to filament 4 g is considered as allodynia and filament 15 g is indicative of hypernociception, while 8 g is an intermediate response (Flatters, S. J. L. and Bennett, G. J. 2004. Pain 109: 150-61).

In a second time, after 10 minutes of rest, the withdrawal response was measured with an electronic von Frey model INSIGHT®, Brazil. The test consists of evoking the withdrawal response by applying a manual force transducer from the electronic analgesiometer that has a 0.5 mm² polypropylene tip. This is applied perpendicular to the central plantar area of the right hind paw with a gradual increase in force. The paw is removed with a clear flinch response after paw withdrawal. The intensity of the pressure is recorded electronically. The response value is averaged over three measurements. The animal is evaluated before and after the treatments and the results are expressed as difference (Δ) of the withdrawal threshold in grams by subtracting the mean of the measurements at the different time intervals from the mean of the measurements at time 0 (Cunha, T M et al. 2004. Braz. J. Med. Biol. Res. 37: 401-407).

### Hot Plate Test

All procedures were performed in a quiet environment with the least possible interaction with the experimenter and gentle handling of the animals to minimize stress-induced analgesia. The animals were gently placed on the electrically heated metal surface of the dish at a constant temperature of 52±0.2 ° C. according to the method described by Eddy and Leimbach in 1953, this includes a plastic cylinder approximately 20 cm in diameter by 28 cm high, to limit its movements (Eddy, N N and Leimbach, D. 1953. J Pharmacol. Exp. Ther. 107: 385-388). In this way, the reaction time or paw withdrawal latency (PWL) was recorded before the noxic caloric stimulus by means of the electronic timer, which starts its activity and stops by means of a manual pedal that the experimenter executes. Withdrawal and shaking, licking of the paw or jumping of the animal was considered a positive response. Exposure will be interrupted after 20 seconds to prevent tissue damage. During the baseline measurements, animals that took more than 20 seconds to respond were excluded from the experiment. Three measurements were made that were averaged.

### Persistent Pain Model (Formalin Test. Spontaneous Behavior)

The rats were individually placed in an open cylindrical glass chamber (34×30×28 cm). The animals were habituated to the chamber for 20 minutes before the injection and returned to it immediately after the injection for observation. One hour prior to the formalin injection, the animals were gently immobilized for the oral administration by gavage of JM-20 or its vehicle according to their assigned group. The formalin (50 μL, s.c.) was injected into the plantar region of the right hind paw of the rat using a 26G needle microsyringe. The times in the previously reported licking/biting behaviors of the injected paw, withdrawals or raised paw after the formalin injection were recorded. Nocidefensive behaviors were observed for 60 minutes using a digital stopwatch for 5-minute observation periods, determining the maintenance time of each of the following 3 behaviors according to the remodified method of Dubuison and Dennis 1977 by Watson (Watson, G S et al. 1997. Pain 70: 53-8). Licking/biting of the injured paw=2, paw elevated from the floor, tips of the digits can be on it=1, neither behavior, any part of the paw other than the tips of the digits is in contact with any surface of the box=0. The weight of the formalin pain scale for each rat was calculated at 12 intervals of 5 minutes during the 60 minutes of observation by the formula: pain scale=[1×(time in sec with elevated inflamed paw)+2×(time in sec with lick/bite of the inflamed paw)]/300 sec.

### Example 2

### Evaluation of Spontaneous Withdrawal Symptoms to Morphine

Animals were evaluated in transparent test boxes individually after a 5-minute adaptation period and observed for 30 minutes. In this case, the number of jumps or escape attempts, and the number of tremors in the hind paws that occurred during the observation period were recorded. In addition, in case of the presence of mastication and piloerection, the scale (1) was assigned and in its absence the scale (0) evaluated every 5 minutes during the total observation period (30 minutes) (Lu, L. et al. 2000 Eur J Neurosci 12: 4398-4404). These are some of the graded symptoms (jumping, paw tremors, wet-dog shakes) and symptoms checked from the Gellet-Hottzman scale that is classically used to evaluate this syndrome, but other authors use some of these isolated symptoms recorded in their studies, as not always all the symptoms and signs are observed in this syndrome (Zharkovsky, A. et al. 1993. Naunyn Schmiedebergs Arch Pharmacol 347 (5): 483-486; Gao, J L et al. 2014. BMC Complementary and Alternative Medicine 14: 308: 1472-688).

### Example 3

### Chronic Constriction Model of the Sciatic Nerve

### Measurement of Behaviors Indicative of Allodynia, Mechanical and Thermal Hypernociception in Tolerant/Hyperalgesic Mononeuropathic Rats

The animals were anesthetized with thiopental (50 mg/Kg, i.p.), after asepsis and antisepsis of the operative region, the common sciatic nerve was exposed laterally. Proximal to its trifurcation, the nerve was released from its tissue adhesions in a segment of 7 to 10 mm and 3 ligatures (chromed 4-0) were tied loosely around the nerve at intervals of 1 to 1.5 mm between them. The ligatures were tied at 40× magnification to prevent constriction and arrest of the circulation through the epineural superficial vascularization. The incision was closed by anatomical planes. We proceeded in a similar manner without performing ligation in the case of sham operated animals (Bennett, G. J. and Xie, Y. K. 1988. Pain 33: 87-107). The mechanical allodynia of the hind paw was assessed by the paw withdrawal response to stimulation of the von Frey filaments. Once the animals were placed in the test boxes (inverted plastic boxes 21×16×27 cm³ with lids), the filaments were applied to the plantar surface of the hind paw (center) in an ascending and descending manner as necessary to close the response threshold. Each filament was applied 5 times, the response 3 out of 5 applications will be considered positive. The lowest stimulus intensity corresponded to 0.25 g and the maximum to 15 g. Based on the response pattern and strength of each filament, 50% of the response threshold was calculated in grams. The animal must have its four paws supported, resting on a metal mesh floor) it cannot be exploring or grooming at the time of measurement, the adaptation period of 5-10 minutes was fulfilled. The resulting pattern of positive or negative response was tabulated using the conversion X=withdrawn 0=not withdrawn and 50% of the response threshold was interpolated using the formula: 50% g threshold=(10 [^{xf+κδ}])/10,000, where xf=value (in Log units) of the final filament used, κ=tabular value for positive or negative pattern and δ=mean of differences between stimulus (in Log units), in this case 0.224 (Chaplan, S R et al. 1994. J. Neurosci. Methods 53: 55-63).

The procedure was similar for the measurement of mechanical hypernociception, but the withdrawal response was measured with an electronic von Frey model INSIGHT®, Brazil. (Cunha, T. M. et al. 2004. Braz. J. Med. Biol. Res. 37: 401-407). The Hargreaves et al. 1988 test was used in CCI rats to direct the caloric stimulus to the medial plantar area of the damaged hind paw. The animals were gently placed in the test boxes on top of the glass floor of the plantar test kit (IITH Life Science model 390G). After an adaptation period of 5 minutes, the light source will be directed from the base of the apparatus towards the area of the skin of the hind paw that rests on the glass floor through the mirror of the source. The heat light source is interrupted with the nocidefensive response and the timer of the equipment records the latency time of the withdrawal. The intensity of the light will be adjusted at the beginning of the experiment according to the average of the baseline measurements with mean values of 10-12 s. Exposure is interrupted after 20 seconds to avoid injury (cut off). The latency to withdrawal defines the threshold for pain response to heat. Then, 3 measurements are run with 5 minutes' rest and the 2 closest measurements are averaged (Hargreaves, K. et al. 1998. Pain 32: 77-88; Coderre, T. J. et al. 2004. Pain 112: 94-105).

### Example 4

### Drug Safety Behavioral Testing

In order to rule out signs of motor impairment, sedation or catalepsy the following test were conducted. Rotating rod test or rota-rod test. The apparatus consists of a 2.5 cm diameter rod divided into 4 compartments. The bar rotates at a constant speed of 22 rpm and the time it took for the animals to fall from the bar was evaluated. The animals were evaluated 1 h after the supply. The cut-off time used was 60 seconds. In addition, the tone, posture, righting reflex, corneal, vital signs and general state of the animal were explored from 30 minutes to 3-hour post-administration of the drugs separately and in combination. The corneal and tympanic reflex were explored using the tip of a fine paper to stimulate the cornea or the auditory canal and the rapid response of opening and closing the eyes or the mobilization of the ears was observed in normal animals. The evaluation of the posture and the righting reflex was based on the Devor and Zalkind scale. Scale for posture: 0=normal posture, grooming on hind paws; 1=moderate atony and ataxia, supports weight, but cannot stand on hind paws; 2=supports your weight, but ataxia is severe; 3=maintains muscle tone, but cannot support your weight, only small movements of intention; 4=atony, flaccidity, totally immobilized, no effort to mobilize. Righting reflex scale: 0=rat struggles when placed horizontally on its back on a table followed by a rapid, coordinated, and powerful reincorporation; 1=Moderate resistance when placed on its back with rapid but not powerful reincorporation; 2=no resistance to being placed on its back, with effort, but finally successful reincorporation; 3=unsuccessful reinstatement; 4=no movements (Rosland, J. H. et al. 1990. Pharmacol. Toxicol. 66: 382-6; Devor, M. and Zalkind, V. 2001. Pain 94: 101-112).

### Example 5

### Drug Interaction Study. Isobolographic Analysis

The method is based on the selection of an effect level that is usually 50% of the maximum effect (ED50%), in case this effect is not produced the ED30 is used, these effects are obtained from the corresponding dose-response curves of each drug administered in 4 doses to the animals. An additive combination corresponding to the determined effect is made up of 2 parts that provide a fraction of said effect. In this case, each fraction was selected to be 0.5 so that the theoretical ED50 (ED50T) of the combination contains 0.5 of the ED50 of morphine and 0.5 of the ED50 of JM-20. Subsequently, an experimental DRC is created, that contains combinations at the established proportion (ED50T) ½, ¼, ⅛ and 1/16 and the experimental ED is calculated, which is statistically compared with the theoretical one. The isobologram shows the results in a graph whose coordinates represent the contribution of each drug. The line that connects the ED50 of both drugs as intercepts contains all possible additive combinations (simple additivity line) and the center of said line corresponds to the ED50T of the combination or additivity point (Tallarida, R J 2011. Genes and Cancer 10: 1003-1008; Raffa, R B et al. 2010. J. Pain 11: 701-709). The resulting experimental point DE50E is plotted in the Cartesian coordinate system and the region where it is located determines the type of interaction. In the case that the interaction is synergistic, the experimental point is located below the additivity line. In the opposite case, if an antagonistic interaction results, the point will be located on the additivity line and if the point is located in a sector close to the additivity line, the interaction will be simple additivity. Furthermore, the interaction index between drugs is calculated according to the following formula: DE50E/DE50T. If the resulting value is less than 1 it corresponds to a synergistic interaction; as it is equal to 1, the interaction is additive, and if it is greater than 1, it is antagonistic. The statistical analysis of the data obtained in the log dose-response curves was analyzed by linear least squares regression to determine the ED50. The statistical parameters relative to the isobolograms were calculated with a computer program from the Pharmacobiology Department of the Center for Research and Advanced Studies, South Headquarters, Mexico, DF. The dose-response curve data were compared with their respective controls by one-way analysis of variance (ANOVA) followed by the Tukey or Dunnett test to compare the differences with the different treatments. Statistical significance between ED5OT and ED5OE was determined by Student's t test, considering in all cases significance at a level of 5% (p<0.05) (Argüelles, C F et al. 2002. Anesthesiology 96: 921-5; Caram-Salas, N L et al. 2006. Pharmacology 77: 53-62).

### Example 6

### Histopathological Study

The animals were sacrificed 14 days post-CCI due to diethyl ether overdose and samples were taken for histopathological study of the sciatic nerve, 5 mm distal to the lesion of the injured paw ligation, which were stored in fixation solution (10 formalin %) and cut with a thickness of 4pm, the staining was carried out with hematoxylin and eosin. We proceeded in a similar way with the sciatic nerve sections of the sham operated animals. These were qualitatively analyzed under optical microscopy (20^{X}) to study the changes induced by CCI. Vehicle-treated animals show increased cellularity relative to sham operated animals as a result of Schwann cell proliferation and macrophage infiltration, presence of digestion chambers in Schwann cells with myelin ovoids, alignment disordered axons with loss of their myelin sheaths indicating Wallerian degeneration. Qualitatively, animals treated with gabapentin as a positive control show a decrease in these alterations. The samples were taken from animals treated with increasing doses of independent drugs and with those of the combination (Sudoh, Y. et al. 2004. Reg. Anesth. Pain Med.29: 434-40; Debový, P. 2011. Annals of Anatomy 193: 267-275).

1. A composition combination comprising:
a) a first composition comprising (3-ethoxycarbonyl-2-methyl-4-(2-nitrophenyl)-4,11-dihydro-1H-pyrido [2,3-b] [1,5] benzodiazepine, which is known as JM20 and
b) a second composition comprising at least one opioid compound.

2. The composition combination according to claim 1, wherein said opioid compound is a natural alkaloid, a synthetic compound or a semisynthetic compound.

3. The composition combination according to claim 2, wherein said opioid compound is selected from the group consisting of:
Phenanthrenes selected from the group consisting of morphine, codeine, hydromorphone, levorphanol, oxycodone, hydrocodone, oxymorphone, buprenorphine, nalbuphine, butorphanol, and heroin;
Benzomorphanes selected from the group consisting of pentazocine, dezocine, phenazocine, and eptazocine;
Phenylpiperidines selected from the group consisting of fentanyl, alfentanil, sufentanil, remifentanil, and meperidine;
Diphenylheptanes selected from the group consisting of methadone and propoxyphene;
tramadol or tapentadol and
a combination thereof.

4. The method of combination therapy according to claim 11, wherein said first composition a) and said second composition b) are administered in the same pharmaceutical form or separately, simultaneously, or sequentially during the course of treatment.

5. The method of combination therapy according to claim 11, wherein said first composition a) and said second composition b) are administered enterally and/or parenterally.

6. The composition combination according to claim 1 comprising said first composition a) and said second composition b) in fixed dose proportions.

7. The composition combination according to claim 6 wherein said first composition a) and said second composition b) are presented in a single pharmaceutical form or packaged together in a single package or in separate packages to be prescribed exclusively with morphine.

8. The method of combination therapy according to claim 11 wherein said administration is therapeutically effective to prevent tolerance and/or hyperalgesia induced by short and long-term exposure to opioids.

9. The method of combination therapy according to claim 11 wherein said administration is therapeutically effective to prevent tolerance and opioid dependence, its withdrawal symptoms and its addictive power.

10. The method of combination therapy according to claim 11 wherein said administration is therapeutically effective for the treatment of morphine-resistant neuropathic pain.

11. A method of combination therapy for treating a patient who can develop opioid-induced neuroadaptations which comprises administering to the patient a therapeutically effective amount of the composition combination according to claim 1 to improve the analgesic efficacy of the opioid and reduce dependency, withdrawal symptoms and addictive power.

12. A method of combination therapy for treating a patient with neuropathic pain who receives treatment with morphine which comprises administering to the patient a therapeutically effective amount of the composition combination according to claim 1 wherein the morphine as said opioid compound is administered between 1.74-30.3 mg/day subcutaneously and said compound JM-20 is administered between 4.06-70.7 mg/day orally to reduce opioid resistance as well as tolerance and/or hyperalgesia facilitated in these conditions.

13. The composition combination according to claim 1 wherein said first composition a) and said second composition b) are present in separate pharmaceutical forms in separate packages.

14. The composition combination according to claim 13 wherein said second composition b) comprises morphine, and said first composition a) and said second composition b) are present in said separate pharmaceutical forms in separate packages.

15. The composition combination according to claim 14 wherein said separate pharmaceutical forms are suitable for enteral and/or parenteral administration.

16. The method of combination therapy according to claim 11 wherein said second composition b) comprises morphine, and said first composition a) and said second composition b) are administered from separate packages.
